(19) United States
(12) Patent Application Publication
(10) Pub. No.: US 2007/0134725 A1
(43) Pub. Date: Jun. 14, 2007

(54) METHOD TO PREDICT RISK OF BPH PROGRESSION

(75) Inventors: Kevin M. Slawin, Houston, TX (US); Michael Kattan, Cleveland Heights, OH (US)

Correspondence Address: SCHWEGMAN, LUNDBERG, WOESSNER & KLUTH, P.A., P.O. BOX 2938, MINNEAPOLIS, MN, 55402, US

(73) Assignees: Baylor College of Medicine; Memorial Sloan-Kettering Cancer Center

(21) Appl. No.: 11/530,763

(22) Filed: Sep. 11, 2006

Related U.S. Application Data
(63) Continuation of application No. PCT/US05/08356, filed on Mar. 11, 2005.
(60) Provisional application No. 60/552,803, filed on Mar. 11, 2004.

(51) Int. Cl. G01N 33/53; G06F 19/00

(52) U.S. Cl. 435/7.1; 702/19

(57) ABSTRACT

A method to predict benign prostatic hyperplasia symptom progression, acute urinary retention, need for surgical intervention and/or prostate cancer development in patients is provided.

### CROSS-REFERENCE TO RELATED APPLICATIONS

This application is a continuation under 35 U.S.C. 111(a) of PCT/US/2005/008356, filed on Mar. 11, 2005, and published in English on Sep. 22, 2005 as WO 2005/088313, which claims the benefit under 35 U.S.C. 119(e) of U.S. Provisional Application Ser. No. 60/552,803, filed Mar. 11, 2004, which applications and publication are incorporated herein by reference.

### BACKGROUND

Benign prostatic hyperplasia (BPH) is the nonmalignant (noncancerous) enlargement of the prostate gland, a common occurrence in older men. It is also known as benign prostatic hypertrophy (BPH) and as nodular hyperplasia of the prostate. As a man matures, the prostate goes through two main periods of growth. The first occurs early in puberty, when the prostate doubles in size. At around age 25, the gland begins to grow again. This second growth phase often results, years later, in BPH. BPH rarely causes symptoms before age 40, but more than half of men in their sixties and as many as 80 percent in their seventies and eighties have some symptoms of BPH.

As the prostate enlarges, the layer of tissue surrounding it stops it from expanding, causing the gland to press against the urethra which courses through the center of the prostate. The bladder wall becomes thicker and irritable. The bladder begins to contract even when it contains small amounts of urine, causing more frequent urination. Eventually, the bladder weakens and loses the ability to empty itself. Urine remains in the bladder. The narrowing of the urethra and partial emptying of the bladder cause many of the problems associated with BPH.

Severe BPH can cause serious problems over time. Urine retention and strain on the bladder can lead to urinary tract infections, bladder or kidney damage, bladder stones, and incontinence. When BPH is found in its earlier stages, there is a lower risk of developing such complications. If the bladder is permanently damaged, treatment for BPH, including drug treatment with, for example, finasteride (Proscar®, Merck & Co., Inc.), dutasteride (Avodart®, GlaxoSmithKline), terazosin (Hytrin®, Abbott Laboratories), doxazosin (Cardura®, Pfizer, Inc.), tamsulosin (Flomax®, Boehringer Ingelheim Pharmaceuticals, Inc.), prazosin (Minipress®, Pfizer, Inc.; generic) or alfuzosin (Uroxatral®, Sanofi-Synthelabo), minimally invasive therapy, including transurethral microwave procedures or transurethral needle ablation, or conventional surgery (surgical intervention), including transurethral surgery, open surgery or laser surgery, may be ineffective.

Therefore, there is a need in the art for nomograms for improved prediction of outcome in patients with BPH disorders, such as patients who are likely to experience acute urinary retention (AUR) or require surgical intervention (SI).

### SUMMARY OF THE INVENTION

The invention provides methods, apparatus and nomograms to predict progression of benign prostatic hyperplasia (BPH) in a patient, with and without drug therapy, including a 5 alpha reductase inhibitor, such as dutasteride or finasteride, an alpha blocker, or other therapy for BPH, or a combination of therapies. In one embodiment, the invention provides methods, apparatus and nomograms to predict whether a patient with BPH will experience acute urinary retention (AUR), require surgical intervention (SI) and/or experience a worsening of BPH symptoms, e.g., within a defined period of time. One embodiment of the invention provides methods, apparatus and nomograms to predict the risk of both the progression of BPH and prostate cancer development.

The methods employ values (scores) for one or more factors, factors including age, prostate volume (PV), maximal flow rate of urine (Qmax), American Urological Association symptom index (AUA-SI) score, BPH impact index (BII) score, BPSA level (or amount), prior alpha blocker use, drug therapy such as non alpha blocker drug therapy or placebo, PSA level (or amount), post void residual urinary volume (PVR), proPSA level (or amount), intact non-complexed PSA level (or amount), JM-27 level (or amount), caveolin-1 level (or amount), caveolin-2 level (or amount), and/or the presence, absence or level of other markers, e.g., markers present in a physiological fluid sample such as a protein found in the blood or markers found in prostate biopsies, to predict patient outcome. In one embodiment, prior alpha blocker use is not a factor. Physiological samples may be collected at any time, including prior to, during or after therapy, such as prior to, during or after drug therapy, minimally invasive therapy, or surgical intervention. Additionally, in methods to predict BPH progression and prostate cancer development, one or more of the following factors may be considered: age, ethnicity, a PSA level, AUA-SI score, BII score, Qmax, PVR, proPSA level, intact non-complexed PSA level, JM-27 level, caveolin-1 level, caveolin-2 level, PV, prior use of alpha blockers, family history of prostate cancer, status of previous biopsies and/or BPSA level. In one embodiment, prior alpha blocker use is not a factor. Additionally, one or more of the following factors may also be considered: the amount or level of VEGF, UPAR, UPA, sVCAM, TGF-β₁, IL6sR, IL6, and/or a Gleason score. The methods may also include factors such as drug therapy, drugs including a 5 alpha reductase inhibitor, such as dutasteride or finasteride, an alpha blocker, or other medical therapy for BPH, or a combination thereof.

In one embodiment, the invention includes a method to predict the risk (probability) of progression of BPH in a patient, with or without drug therapy, including detecting or determining values for a plurality of factors comprising age, PSA level, PV, Qmax, PVR, AUA-SI score, BII score, BPSA level, non alpha blocker BPH drug therapy, and/or prior use of alpha blockers; and correlating the values for age, PSA level, PV, Qmax, PVR, AUA-SI score, BII score, BPSA level, non alpha blocker BPH drug therapy, and/or prior use of alpha blockers with the risk or probability of progression of BPH. In one embodiment, the plurality of factors is three or more, four or more, five or more, six or more, or seven or more, factors. In one embodiment, the factors include age, PVR and BII score, and optionally non alpha blocker BPH drug therapy, PSA level and/or PV. In another embodiment, the factors include age, AUA-SI score, BII score, Qmax, and non alpha blocker BPH drug therapy, and optionally PVR and/or volume of transition zone of the prostate. In another embodiment, the factors include BII score, PV, PSA level, Qmax, non alpha blocker BPH drug therapy and prior alpha blocker use, and optionally AUA-SI score.

Another embodiment of the invention includes a method to predict the risk of progression of BPH in a patient, with or without drug therapy, including detecting or determining one or more of the following factors: age, PSA level, AUA-SI score, BII score, Qmax, PV, PVR and/or BPSA level; and correlating the amount, level or score of the factors with the risk of BPH progression.

One embodiment of the invention includes a method to determine the risk of progression of BPH in a patient, with or without drug therapy, including detecting or determining one or more of the following factors: age, ethnicity, PSA level, AUA-SI score, BII score, Qmax, PVR, proPSA level, intact non-complexed PSA level, JM-27 level, caveolin-1 level, caveolin-2 level, prostate volume, prior use of alpha blockers and/or BPSA level; and correlating the amount, level or score of the factors with the risk of BPH progression. In one embodiment, prior alpha blocker use is not a factor.

Additionally, methods to predict the risk of BPH progression and prostate cancer development in a patient, with or without drug therapy, is provided. According to the method, one or more of the following factors may be considered: age, ethnicity, PSA level, AUA-SI score, BII score, Qmax, PVR, proPSA level, intact non-complexed PSA level, JM-27 level, caveolin-1 level, caveolin-2 level, PV, prior use of alpha blockers, family history of prostate cancer, status of previous biopsies and/or BPSA level. Additionally, one or more of the following the factors may also be considered: the amount or level of VEGF, UPAR, UPA, sVCAM, TGF-β₁, IL6sR, IL6, and/or a Gleason score.

Thus, the methods may also include factors such as drug therapy, drugs including a 5 alpha reductase inhibitor, such as dutasteride or finasteride, or a alpha blocker, or other medical therapies for BPH, or a combination thereof. In one embodiment, prior alpha blocker use is not a factor.

The invention also provides an apparatus. The apparatus includes a data input means, for input of information for a plurality of factors; a processor, executing a software for analysis of the information; wherein the software analyzes the information and provides the risk of BPH progression in the mammal. In one embodiment, the plurality of factors is selected from age, PSA level, PV, Qmax, PVR, AUA-SI score, BII score, BPSA level, non alpha blocker BPH drug therapy, and/or prior use of alpha blockers, and the processor executes a software for analysis of information and the software analyzes the information and provides the probability of BPH symptom progression, AUR and/or SI, e.g., within a specified time, for instance, within 1, 2, 3, 4 or more years, in the mammal. In another embodiment, the information includes a plurality of the following factors: age, PSA level, AUA-SI score, BII score, Qmax, PV, PVR, and/or BPSA level. In yet another embodiment of the invention, the test information includes one or more of the following factors: age, ethnicity, PSA level, AUA-SI score, BII score, Qmax, PVR, proPSA level, intact non-complexed PSA level, JM-27 level, caveolin-1 level, caveolin-2 level, PV, current or future drug therapy, prior use of alpha blockers, and/or BPSA level. Additionally, information may include one or more of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of alpha blockers, family history of prostate cancer, status of previous biopsies and/or BPSA level, to determine the risk of BPH progression and prostate cancer development. One or more of the following the factors may also be considered: the amount or level of VEGF, UPAR, UPA, sVCAM, TGF-β₁, IL6sR, IL6, and/or a Gleason score. Other factors may include drug therapy, drugs including a 5 alpha reductase inhibitor, such as dutasteride or finasteride, or an alpha blocker, or other medical therapies for BPH, or a combination thereof. In one embodiment, prior alpha blocker use is not a factor.

The invention also provides a method to predict the risk or probability of BPH progression, AUR and/or SI, in a patient, with and without drug therapy. In one embodiment, the method includes inputting information to a data input means, wherein the information comprises a plurality of factors including age, PSA level, PV, Qmax, PVR, AUA-SI score, BII score, non alpha blocker BPH drug therapy, and/or prior use of alpha blockers, of a patient; executing a software for analysis of the information; and analyzing the information so as to provide the risk of BPH progression, AUR and/or SI in the patient, e.g., within the next 2 years. In other embodiment, the information includes one or more of, e.g., a plurality of, the following factors: age, PSA level, AUA-SI score, BII score, Qmax, PV, PVR and/or BPSA level. In another embodiment, the information includes a plurality of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of alpha blockers and/or BPSA level of a BPH patient. Additionally, information may include one or more of the following factors: age, ethnicity, PSA level, AUA-SI score, BII score, Qmax, PVR, proPSA level, intact, non-complexed PSA level, JM-27 level, caveolin-1 level, caveolin-2 level, PV, prior use of alpha blockers, family history of prostate cancer, status of previous biopsies and/or BPSA level to determine the risk of BPH progression and prostate cancer development. One or more of the following the factors may also be employed in the method: the amount or level of VEGF, UPAR, UPA, sVCAM, TGF-β₁, IL6sR, IL6, and/or a Gleason score. Other factors may include drug therapy, including a 5 alpha reductase inhibitor, such as dutasteride or finasteride, or alpha blocker therapy, or other medical therapies for BPH or a combination thereof. In one embodiment, prior alpha blocker use is not a factor.

The invention also provides a nomogram that may employ one or more clinical and pathological measures of BPH, as well as one or more serum/plasma proteins, including, but not limited to, one or more factors including age, PSA level, Qmax, AUA-SI score, PVR, BII score, BPSA level, PV, non alpha blocker BPH drug therapy (or placebo), prior use of an alpha blocker, proPSA level, intact non-complexed PSA level, JM-27 level, caveolin-1 level and/or caveolin-2 level, to predict outcomes in clinical situations for BPH patients, including an AUR experience, requirement for SI and/or a worsening the symptoms of BPH. Additionally, the one or more factors may include a plurality of the following factors: age, ethnicity, PSA level, AUA-SI score, BII score, Qmax, PVR, proPSA level, intact, non-complexed PSA level, JM-27 level, caveolin-1 level, caveolin-2 level, PV, prior use of alpha blockers, family history of prostate cancer, status of previous biopsies and/or BPSA level to determine the risk of BPH progression and prostate cancer development. One or more of the following the factors may also be considered: the amount or level of VEGF, UPAR, UPA, sVCAM, TGF-β₁, IL6sR, IL6, and/or a Gleason score. The nomogram may also include factors such as drug therapy, including a 5 alpha reductase inhibitor, such as dutasteride or finasteride, or alpha blocker therapy, or other medical therapies for BPH, or a combination thereof. In one embodiment, prior alpha blocker use is not a factor.

The invention also includes the use of nomograms to predict the prognosis of a BPH patient, such as an AUR experience, requirement of SI and/or a worsening of the symptoms of BPH. Nomograms may include markers present in physiological fluids and tissues as well as standard clinical parameters.

The invention also provides a method to predict the probability of progression of BPH in a patient. The method comprises correlating a plurality of the following factors: age, ethnicity, PSA level, AUA-SI score, BII score, Qmax, PV, PVR, proPSA level, intact non-complexed PSA level, JM-27 level, caveolin-1 level, caveolin-2 level, prior use of alpha blockers, and/or BPSA level obtained from the patient, with the risk BPH progression, including an AUR experience, SI requirement and/or a worsening of the symptoms of BPH. Additionally, the one or more factors may include one or more of the following factors: age, ethnicity, PSA level, AUA-SI score, BII score, Qmax, PVR, PV, proPSA level, intact non-complexed PSA level, JM-27 level, caveolin-1 level, caveolin-2 level, PV, prior use of alpha blockers, family history of prostate cancer, status of previous biopsies and/or BPSA level, to determine the risk of BPH progression and prostate cancer development. One or more of the following the factors may also be considered: the amount or level or VEGF, UPAR, UPA, sVCAM, TGF-β₁, IL6sR, IL6, and/or a Gleason score. In one embodiment, drug therapy, including a 5 alpha reductase inhibitor, such as dutasteride or finasteride, or alpha blocker therapy, or other medical therapy for BPH, or a combination thereof, may be a factor. In one embodiment, prior alpha blocker use is not a factor.

In one embodiment, the invention provides a method to predict the risk BPH progression and the risk of prostate cancer development in a patient. The method employs a plurality of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of alpha blockers, family history of prostate cancer, status of previous biopsies and/or BPSA level, the levels, values or scores of which are correlated with the risk of progression of BPH and/or the risk of prostate cancer development. In one embodiment, additional factors are employed, e.g., VEGF, UPAR, UPA, sVCAM, TGF-β₁, IL6sR, and/or IL6 levels, a Gleason score, drug therapy such as 5 alpha reductase inhibitor therapy, or alpha blocker therapy, or a combination of such therapies. In one embodiment, the prior use of alpha blockers is not a factor. Also provided is an apparatus, which includes a data input means, for input of information comprising detecting or determining a plurality of the factors described above, a processor, executing a software for analysis of the information; wherein the software analyzes the information, and provides the risk of BPH progression and prostate cancer development in the mammal. Further provided is a method to determine BPH progression and prostate cancer development in a patient. The method includes inputting test information to a data input means, wherein the information comprises one or more of the factors described above executing a software for analysis of the test information; and analyzing the test information so as to provide the risk of BPH progression and prostate cancer development in the patient. Also provided is a nomogram for the graphic representation of a quantitative probability that a patient will experience BPH progress and development of prostate cancer. The nomogram includes a plurality of scales and a solid support, the plurality of scales being disposed on the support and comprising one or more scales for one or more of the factors described above, a points scale, a total points scale and one or more predictor scales. The scales for each factor has values on the scales, and the scales for each factor are disposed on the solid support with respect to the points scale so that each of the values of the factors can be correlated with values on the points scale. The total points scale has values on the total points scale, and the total points scale is disposed on the solid support with respect to the predictor scale so that the values on the total points scale may be correlated with values on the predictor scale. The values on the points scale correlating with the patient's factors are added together to yield a total points value, and the total points value are correlated with the predictor scale to individually predict the quantitative probability of BPH progression and prostate cancer development.

Thus, the invention provides a method for predicting the probability of BPH progression and prostate cancer development in a patient. The method includes detecting or determining one or more of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of alpha blockers, family history of prostate cancer, status of previous biopsies and/or BPSA level; and correlating the amount, level or score of the factors with the probability of progression of BPH and prostate cancer development in the patient.

Also provided is a method to predict the prognosis of a BPH patient. The method includes determining a set of factors for a patient, which set comprises one or more of the following factors: age, ethnicity, PSA level, AUA-SI score, BII score, Qmax, PVR, proPSA level, intact non-complexed PSA level, JM-27 level, caveolin-1 level, caveolin-2 level, prior use of alpha blockers and/or BPSA level; matching the factors to the values on the scales of a nomogram, determining a separate point value for each of the factors; adding the separate point values together to yield a total points value; and correlating the total points value with a value on the predictor scale of the nomogram to determine the prognosis of the BPH patient.

### BRIEF DESCRIPTION OF THE FIGURES

FIG. 1. Exemplary nomograms to predict the probability of acute urinary retention (AUR) or surgical intervention (SI) in BPH patients within two years.

FIG. 2. Graph of nomogram prediction versus actual outcomes of patients.

FIG. 3. Nomogram to predict the probability of AUR or SI in BPH patients within four years.

FIG. 4. Nomogram to predict symptom progression in BPH patients within four years.

FIG. 5. Exemplary embodiment of a nomogram system architecture.

### DETAILED DESCRIPTION OF THE INVENTION

The invention includes a method to predict BPH progression in a patient. In one embodiment, the method is particularly useful for evaluating patients at risk for an AUR experience, SI and/or a worsening of symptoms of BPH. Specifically, the detection or determination of one or more of the following factors: age, ethnicity, PSA level, AUA-SI score, BII score, Qmax, PVR, PV, proPSA level, intact non-complexed PSA level, JM-27, caveolin-1, caveolin-2, prior use of alpha blockers, BPSA level, and/or yet other markers for BPH, may be useful in predicting BPH progression, for example, the risk of an AUR experience, SI, and/or the worsening of one or more symptoms of BPH, e.g., a 4 point or greater increase in AUA-SI score. The invention also includes a method to predict the reduction of the risk of BPH progression with the use of drug therapy, including a 5 alpha reductase inhibitor, such as dutasteride or finasteride, an alpha blocker, or other medical therapy for BPH, or a combination thereof. In one embodiment, prior alpha blocker use is not a factor.

The invention further includes a method to predict the risk of both BPH progression and the risk of developing prostate cancer, with and without drug therapy. Specifically the method includes the detection or determination of one or more of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of alpha blockers, family history of prostate cancer, status of previous biopsies and/or BPSA level (or amount), to determine the risk of BPH progression and prostate cancer development. One or more of the following the factors may also be considered: the level or amount of VEGF, UPAR, UPA, sVCAM, TGF-β₁, IL6sR, IL6, and/or a Gleason score. The methods may also include drug therapy, including a 5 alpha reductase inhibitor, such as dutasteride or finasteride, an alpha blocker or other medical therapy for BPH, or a combination thereof, as a factor. In one embodiment, prior alpha blocker use is not a factor. The invention also includes a method to predict the reduction of the risk of both BPH progression and prostate cancer development with the use of drug therapy, including a 5 alpha reductase inhibitor, such as dutasteride or finasteride, an alpha blocker or other medical therapy for BPH, or a combination thereof, as a factor. In one embodiment, prior alpha blocker use is not a factor.

Definitions

As used herein, “AUA-SI” or “AUA symptom index” refers to a symptom index developed by the American Urological Association (AUA) to categorize enlarged prostate symptoms. The index contains seven questions intended to classify the severity of enlarged prostate symptoms and can be found at http://bphrelief.com/about/symptom index.asp. The questions include within the last month or so 1) how often have you had a sensation of not emptying your bladder completely after you finished urinating; 2) how often have you had to urinate again less than two hours after you finished urinating; 3) how often have you stopped and started again several times when you urinated; 4) how often have you found it difficult to postpone urination; 5) how often have you had a weak urinary stream; and 6) how often have you had to push or strain to begin urination? The answers are selected from not at all (0 score), less than 1 time in 5 (1 as a score), less than ½ the time (2 as a score), about ½ the time (3 as a score), more than ½ the time (4 as a score) and almost always (5 as a score). The other question is, in the last month, how many times did you most typically get up to urinate from the time you went to bed at night until the time you got up in the morning (score is the number of times)? The cumulative score of each of those questions is the AUA-SI score, which may then be compared to a severity scale: 1-7=mild, 8-19=moderate and 20-35 severe.

As used herein, “AUR” or “acute urinary retention” refers to the inability to urinate, causing pain and discomfort. Causes can include an obstruction in the urinary system, stress or neurologic problems.

As used herein, “SI” or “surgical intervention” refers to surgery required to correct and/or relieve one or more symptoms caused by BPH.

As used herein, “BII” refers to the BPH impact index (BII), which measures the health impact of symptoms (available at http://www.mapi-research-inst.com/dhoi.asp).

As used herein, the term “alpha blockers” refers to any drug or other substance that blocks chemical activity (antagonist) at the alpha receptors, sites that respond to adrenaline-like substances, e.g., doxazosin (Cardura®, Pfizer, Inc.) or terazosin (Hytrin®, Abbott Laboratories). An alpha blocker may also be referred to as alpha adrenergic antagonist, alpha adrenergic blocking agent or alpha adrenergic blocker. Prior use of alpha blockers is preferably reported as yes or no. Alternatively, it may be ranked as positive or negative, or absent or present.

As used herein, “prostate volume” or “PV” refers to size and weight of the prostate. Prostate volume is a predictor of both progression and response to 5 alpha reductase inhibitor therapy in patients with BPH. Prostate volume can also aid in the prediction of AUR (Jacobsen et al. 1999).

As used herein, “benign prostatic hyperplasia (BPH) progression” refers to the progression of BPH, which includes, but is not limited to, an increase in prostate size/prostate volume, increase in AUA-SI score, a worsening of one or more symptoms of BPH as manifested by an increase in AUA symptom score of 4 or more over time, increase in BII score, incontinence, urinary tract infection (UTI), increase in PSA (prostate specific antigen) level, increase in BPSA (benign PSA) level, reduction in Qmax (maximal flow rate of urine), AUR experience, bladder damage, kidney damage, bladder stones, increase in PVR (post-void residual urine volume), and/or a need for SI, minimal invasive therapy or drug therapy.

As used herein, “PSA” refers to prostate-specific antigen. PSA is a protein produced by the prostate. An increased amount of PSA in the blood is linked to men who have prostate cancer, benign prostatic hyperplasia or an infection of the prostate gland. A blood sample is measured in an assay and the amount of PSA is reported as ng/ml.

As used herein, “Qmax” refers to maximal flow rate of urine.

As used herein, “BPSA” or “benign PSA” refers to a specific molecular form of free prostate-specific antigen that is found predominantly in the transition zone of patients with nodular benign prostatic hyperplasia. (Mikolajczyk et al. 2000; U.S. Pat. No. 6,482,599). BPSA has been shown to be elevated in patients with BPH (Linton 2003). BPSA is also present in the serum.

As used herein, “proPSA” refers to the form of PSA that in normal prostate glands is secreted into the glandular lumen where seven amino acids are cleaved to create active PSA. There are several isoforms of proPSA (i.e., −2, −4 and −7 proPSA).

As used herein, “free PSA” (fPSA) refers to the various proPSA isoforms, intact free PSA and BPSA

Serum PSA that is measurable by current clinical immunoassays exists primarily as either the free “noncomplexed” form or as a complex with ACT (a₁-antichymotrypsin; Lilja et al. 1991; Stenman et al. 1991). As used herein, “intact, non-complexed PSA” refers to the free noncomplexed form of PSA described above.

As used herein, “JM-27” refers to a gene that is up-regulated in prostate cancer and in symptomatic but not asymptomatic BPH (Prakash et al., 2002). The gene has homology to a family of MAGE/GAGE-like proteins that contain RGD motifs.

As used herein “caveolae” refers to specialized domains of the plasma membrane that are implicated in the sequestration of a variety of lipid and protein molecules. It has been suggested that these important cellular organelles have a pivotal role in such diverse biochemical processes as lipid metabolism, growth regulation, signal transduction, and apoptosis. Caveolin interacts with and regulates heterotrimeric G-proteins. Currently, there are three members of the caveolin multigene family which are known to encode 21-24 kDa integral membrane proteins that comprise the major structural component of the caveolar membrane in vivo. “Caveolin-2” protein is abundantly expressed in fibroblasts and differentiated adipocytes, smooth and skeletal muscle, and endothelial cells. The expression of “caveolin-1” is similar to that of “caveolin-2” while “caveolin-3” expression appears to be limited to muscle tissue types.

As used herein, “PVR” refers to post-void residual urine volume.

As used herein, a sample of “physiological body fluid” includes, but is not limited to, a sample of blood, plasma, serum, seminal fluid, urine, saliva, sputum, semen, pleural effusions, bladder washes, bronchioalveolar lavages, cerebrospinal fluid and the like.

The terms “correlation,” “correlate” and “correlating” include a statistical association between factors and outcome, and may or may not be equivalent to a calculation of a statistical correlation coefficient.

As used herein, “prior alpha blocker use” refers use of alpha blockers at any time in the past up to the present (the time of prediction/determination).

As used herein, “drug therapy” includes therapy that starts at the time of the prediction/determination. Specifically, drug therapy may include a 5 alpha reductase inhibitor, such as dutasteride or finasteride, an alpha blocker, or other medical therapy for BPH, or a combination thereof. Drug therapy may also include the use of multiple drugs within each class, such as the use of two 5 alpha reductase inhibitors

Description

Non-invasive prognostic assays are provided by the invention which detect and/or quantitate markers such as BPSA, proPSA, intact, non-complexed PSA, JM-27, caveolin-1, caveolin-2, or PSA levels in the body fluids or tissue biopsies as well as other measures of BPH progression of mammals, including humans, factors including age, ethnicity, AUA-SI score, BII score, Qmax, PVR, PV, and/prior drug therapy. Other non-invasive assays may detect or quantitate levels of VEGF, UPAR, UPA, sVCAM, TGF-β₁, IL6sR, and/or IL6. Such assays may be useful in the prognosis of BPH and/or prostate cancer development. Moreover, such assays provide valuable means of monitoring the status of the BPH and/or prostate cancer development. In addition to improving prognostication, knowledge of the disease status allows the attending physician to select the most appropriate therapy for the individual patient. For example, patients with a high likelihood of an AUR experience, SI and/or prostate cancer development can be treated and monitored closely.

The body fluids that are of particular interest as physiological samples in assaying for BPSA, proPSA, intact non-complexed PSA, JM-27, caveolin-1, caveolin-2, VEGF, UPAR, UPA, sVCAM, TGF-β₁, IL6sR, IL6 or PSA according to the methods of this invention include blood, blood serum, semen, saliva, sputum, urine, blood plasma, pleural effusions, bladder washes, bronchioalveolar lavages, and cerebrospinal fluid. Blood, serum and plasma are preferred, and plasma, such as platelet-poor plasma, is the more preferred sample for use in the methods of this invention. Furthermore, tissue biopsies are also useful for assaying for proteins and/or genes of interest.

Exemplary means for detecting and/or quantitating BPSA, proPSA, intact non-complexed PSA, JM-27, caveolin-1, caveolin-2, VEGF, UPAR, UPA, sVCAM, TGF-β₁, IL6sR, IL6 or PSA levels in mammalian body fluids include affinity chromatography, Western blot analysis, immunoprecipitation analysis, and immunoassays, including ELISAs (enzyme-linked immunosorbent assays), RIA (radioimmunoassay), competitive EIA or dual antibody sandwich assays. In such immunoassays, the interpretation of the results is based on the assumption that the BPSA, proPSA, intact non-complexed PSA, JM-27, caveolin-1, caveolin-2, VEGF, UPAR, UPA, sVCAM, TGF-β₁, IL6sR, IL6 or PSA binding agent, e.g., a BPSA, proPSA, intact non-complexed PSA, JM-27, caveolin-1, caveolin-2, VEGF, UPAR, UPA, sVCAM, TGF-β₁, IL6sR, IL6 or PSA specific antibody, will not cross-react with other proteins and protein fragments present in the sample that are unrelated to BPSA, proPSA, intact, non-complexed PSA, JM-27, caveolin-1, caveolin-2, VEGF, UPAR, UPA, sVCAM, TGF-β₁, IL6sR, IL6 or PSA. Preferably, the method used to detect BPSA, proPSA, intact non-complexed PSA, JM-27, caveolin-1, caveolin-2, VEGF, UPAR, UPA, sVCAM, TGF-β₁, IL6sR, IL6 or PSA levels employs at least one BPSA, proPSA, intact non-complexed PSA, JM-27, caveolin-1, caveolin-2, VEGF, UPAR, UPA, sVCAM, TGF-β₁, IL6sR, IL6 or PSA specific binding molecule, e.g., an antibody or at least a portion of the ligand for any of those molecules. Immunoassays are a preferred means to detect BPSA, proPSA, intact non-complexed PSA, JM-27, caveolin-1, caveolin-2, or VEGF, UPAR, UPA, sVCAM, TGF-β₁, IL6sR, IL6 PSA. Representative immunoassays involve the use of at least one monoclonal or polyclonal antibody to detect and/or quantitate BPSA, proPSA, intact non-complexed PSA, JM-27, caveolin-1, caveolin-2, VEGF, UPAR, UPA, sVCAM, TGF-β₁, IL6sR, IL6 or PSA in the body fluids of mammals. The antibodies or other binding molecules employed in the assays may be labeled or unlabeled. Unlabeled antibodies may be employed in agglutination; labeled antibodies or other binding molecules may be employed in a wide variety of assays, employing a wide variety of labels.

Suitable detection means include the use of labels such as radionucleotides, enzymes, fluorescers, chemiluminescers, enzyme substrates or co-factors, enzyme inhibitors, particles, dyes and the like. Such labeled reagents may be used in a variety of well known assays. See for example, U.S. Pat. Nos. 3,766,162, 3,791,932, 3,817,837, and 4,233,402.

Still further, in, for example, a competitive assay format, labeled BPSA, proPSA, intact non-complexed PSA, JM-27, caveolin-1, caveolin-2, VEGF, UPAR, UPA, sVCAM, TGF-β₁, IL6sR, IL6 or PSA peptides and/or polypeptides can be used to detect and/or quantitate BPSA, proPSA, intact non-complexed PSA, JM-27, caveolin-1, caveolin-2, VEGF, UPAR, UPA, sVCAM, TGF-β₁, IL6sR, IL6 or PSA, respectively, in mammalian body fluids and/or tissue. Also, alternatively, as a replacement for the labeled peptides and/or polypeptides in such a representative competitive assay, labeled anti-idiotype antibodies that have been prepared against antibodies reactive with BPSA, proPSA, intact non-complexed PSA, JM-27, caveolin-1, caveolin-2, VEGF, UPAR, UPA, sVCAM, TGF-β₁, IL6sR, IL6 or PSA can be used.

For example, BPSA, proPSA, intact non-complexed PSA, JM-27, caveolin-1, caveolin-2, VEGF, UPAR, UPA, sVCAM, TGF-β₁, IL6sR, IL6 or PSA levels may be detected by an immunoassay such as a “sandwich” enzyme-linked immunoassay (see Dasch et al. 1990; Danielpour et al. 1989; Danielpour et al. 1990; Lucas et al. 1990; Thompson et al. 1989; and Flanders et al. 1989). A physiological fluid is contacted with at least one antibody specific for BPSA, proPSA, intact non-complexed PSA, JM-27, caveolin-1, caveolin-2, VEGF, UPAR, UPA, sVCAM, TGF-β₁, IL6sR, IL6 or PSA to form a complex with said antibody and BPSA, proPSA, intact non-complexed PSA, JM-27, caveolin-1, caveolin-2, VEGF, UPAR, UPA, sVCAM, TGF-β₁, IL6sR, IL6 or PSA. Then the amount of BPSA, proPSA, intact non-complexed PSA, JM-27, caveolin-1, caveolin-2, VEGF, UPAR, UPA, sVCAM, TGF-β₁, IL6sR, IL6 or PSA in the sample is measured by measuring the amount of complex formation.

Representative of one type of ELISA test is a format wherein a solid surface, e.g., a microtiter plate, is coated with antibodies to BPSA, proPSA, intact non-complexed PSA, JM-27, caveolin-l, caveolin-2, VEGF, UPAR, UPA, sVCAM, TGF-β₁, IL6sR, IL6 or PSA and a sample of a patient's plasma is added to a well on the microtiter plate. After a period of incubation permitting any antigen to bind to the antibodies, the plate is washed and another set of BPSA, proPSA, intact non-complexed PSA, JM-27, caveolin-1, caveolin-2, VEGF, UPAR, UPA, sVCAM, TGF-β₁, IL6sR, IL6 or PSA antibodies, e.g., antibodies that are linked to a detectable molecule such as an enzyme, is added, incubated to allow a reaction to take place, and the plate is then rewashed. Thereafter, enzyme substrate is added to the microtiter plate and incubated for a period of time to allow the enzyme to catalyze the synthesis of a detectable product, and the product, e.g., the absorbance of the product, is measured.

It is also apparent to one skilled in the art that a combination of antibodies to BPSA, proPSA, intact non-complexed PSA, JM-27, caveolin-1, caveolin-2, VEGF, UPAR, UPA, sVCAM, TGF-β₁, IL6sR, IL6 or PSA can be used to detect and/or quantitate the presence of BPSA, proPSA, intact non-complexed PSA, JM-27, caveolin-1, caveolin-2, VEGF, UPAR, UPA, sVCAM, TGF-β₁, IL6sR, IL6 or PSA in the body fluids of patients. In one such embodiment, a competition immunoassay is used, wherein BPSA, proPSA, intact, non-complexed PSA, JM-27, caveolin-1, caveolin-2, VEGF, UPAR, UPA, sVCAM, TGF-β₁, IL6sR, IL6 or PSA is labeled, and a body fluid is added to compete the binding of the labeled BPSA, proPSA, intact non-complexed PSA, JM-27, caveolin-1, caveolin-2, VEGF, UPAR, UPA, sVCAM, TGF-β₁, IL6sR, IL6 or PSA to antibodies specific for BPSA, proPSA, intact non-complexed PSA, JM-27, caveolin-1, caveolin-2, VEGF, UPAR, UPA, sVCAM, TGF-β₁, IL6sR, IL6 or PSA. Such an assay could be used to detect and/or quantitate BPSA, proPSA, intact non-complexed PSA, JM-27, caveolin-1, caveolin-2, VEGF, UPAR, UPA, sVCAM, TGF-β₁, IL6sR, IL6 or PSA.

Thus, once binding agents having suitable specificity have been prepared or are otherwise available, a wide variety of assay methods are available for determining the formation of specific complexes. Numerous competitive and non-competitive protein binding assays have been described in the scientific and patent literature and a large number of such assays are commercially available. Exemplary immunoassays which are suitable for detecting a serum antigen include those described in U.S. Pat. Nos. 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876.

The methods of the invention may be employed with other measures of prostate biology to better predict BPH progression and/or prostate cancer development. For example, the following clinical and pathological criteria (factors) may be used, e.g., age, ethnicity, PV, AUA-SI, BII, Qmax, PVR, prior use of alpha blockers, drug therapy, including a 5 alpha reductase inhibitor therapy, such as dutasteride or finasteride, an alpha blocker therapy or other medical therapy for BPH, or a combination thereof, family history of prostate cancer, status of previous biopsies, Gleason score and/or PSA levels, although the use of other criteria or criteria which can replace one or more or those criteria does not depart from the scope and spirit of the invention. Additionally, once BPH progression and/or prostate cancer development has been predicted, the determination of one or more the following factors: age, ethnicity, PSA level, AUA-SI score, BII score, Qmax, PVR, proPSA level, intact non-complexed PSA level, JM-27 level, caveolin-1 level, caveolin-2 level, PV, family history of prostate cancer, status of previous biopsies, Gleason score, prior use of alpha blockers and/or BPSA level, along with the consideration of drug therapy, can result in the prediction of a reduction in the risk of BPH progression and/or a reduction in the risk of prostate cancer development.

Exemplary Methods, Apparatus and Nomograms without 5α-Reductase Inhibitor Therapy

The present invention provides methods, apparatus and nomograms to predict the risk of BPH progression to aid patients in their treatment of BPH. In one embodiment, a nomogram predicts BPH progression without drug therapy, including the probability of a patient experiencing an AUR, requiring SI or experiencing a worsening in one or more symptoms of BPH, to assist the physician in treating the patient.

One embodiment of the invention is directed to a method to predict the risk of BPH progression in a patient, specifically in a patient not undergoing drug therapy, while another embodiment of the invention is directed to predicting the probability of progression of BPH in a patient undergoing drug therapy, e.g., drug therapy other than with a 5 alpha reductase inhibitor. The methods include detecting or determining a plurality of factors comprising age, PSA level, PV, Qmax, AUA-SI score, BII score, PVR, drug therapy, e.g., non alpha blocker BPH drug therapy, and/or prior use of an alpha blocker; and correlating the amount, level or score of the plurality of factors comprising age, PSA level, PV, Qmax, AUA-SI score, BII score, PVR, drug therapy, e.g., non alpha blocker BPH drug therapy, and/or prior use of an alpha blocker(s) with the risk, or with the probability, of progression of BPH without therapy. In another embodiment, the factors include one more of the following factors: age, PSA level, AUA-SI score, BII score, Qmax, PV, PVR and/or BPSA level. In one embodiment, the factors include one more of the following factors: factors: age, ethnicity, PSA level, AUA-SI score, BII score, Qmax, PVR, proPSA level, intact non-complexed PSA level, JM-27 level, caveolin-1 level, caveolin-2 level, PV, prior use of an alpha blocker(s) and/or BPSA level. In one embodiment, prior alpha blocker use is not a factor.

In one embodiment, the correlating may be accomplished by computer. In one embodiment, the correlating includes accessing a memory storing the selected set of factors. In another embodiment, the correlating includes generating a functional representation and displaying the functional representation on a display. In one embodiment, the displaying includes transmitting the functional representation from a source. In one embodiment, the correlating is executed by a processor or a virtual computer program or interactive web site. In another embodiment, the method further comprises transmitting the quantitative probability of BPH progression. In yet another embodiment, the method further comprises inputting the identical set of factors for the patient within an input device. In another embodiment, the method further comprises storing any of the set of factors to a memory or to a database.

Another embodiment of the invention is directed to an apparatus for predicting the probability of the risk of BPH progression in a BPH patient. The apparatus comprises a data input means, for input of test information comprising detecting or determining one or more of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of alpha blockers and/or BPSA level, a processor, executing a software for analysis of the amount, level or score of one or more of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of alpha blockers and/or BPSA level; wherein the software analyzes the amount, level or score of one or more of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of alpha blockers and/or BPSA level, and provides the risk of BPH progression in the mammal without drug therapy.

Another embodiment of the invention is directed to a nomogram. The nomogram may be generated with a Cox proportional hazards regression model (Cox 1972). Alternatively, the nomogram may be generated with a neural network model (Rumelhart et al. 1986). In another embodiment, the nomogram is generated with a recursive partitioning model (Breiman et al. 1984). In yet another embodiment, the nomogram is generated with support vector machine technology (Cristianni et al. 2000). Other models known to those skilled in the art may alternatively be used. In one embodiment, the invention includes the use of software that implements Cox regression models or support vector machines to BPH progression.

The nomogram may be a graphic representation of a probability that a BPH patient not undergoing 5 alpha reductase therapy will experience a risk of BPH progression, e.g., risk of AUR and/or SI, comprising a set of indicia on a solid support, the indicia comprising one or more factor lines including an age line, an ethnicity line, a PSA level line, an AUA-SI score line, a BII score line, a Qmax line, a PVR line, a proPSA level line, an intact non-complexed PSA level line, a JM-27 level line, a caveolin-1 level line, a caveolin-2 level line, a PV line, a prior use of an alpha blocker(s) line and/or a BPSA level line, a points line, a total points line and a predictor line, wherein the age line, ethnicity line, PSA level line, AUA-SI score line, BII score line, Qmax line, PVR line, proPSA level line, intact non-complexed PSA level line, JM-27 level line, caveolin-1 level line, caveolin-2 level line, PV line, prior use of an alpha blocker(s) line and/or BPSA level line each have values on a scale which can be correlated with values on a scale on the points line, and wherein said total points line has values on a scale which may be correlated with values on a scale on the predictor line, such that the value of each of the points correlating with the patient's age, ethnicity, PSA level, AUA-SI score, BII score, Qmax, PVR, proPSA level, intact non-complexed PSA level, JM-27, caveolin-1, caveolin-2, PV, prior use of alpha blockers and/or BPSA level can be added together to yield a total points value, and the total points value can be correlated with the predictor line to predict the probability that a BPH patient will experience a risk of BPH progression without drug therapy. The solid support may assume any appropriate form such as, for example, a laminated card. Any other suitable representation, picture, depiction or exemplification may be used.

The nomogram may assume any form, such as a computer program, e.g., in a hand-held device, world-wide-web page, e.g., written in FLASH, or a card, such as a laminated card. Any other suitable representation, picture, depiction or exemplification may be used. The nomogram may comprise a graphic representation and/or may be stored in a database or memory, e.g., a random access memory, read-only memory, disk, virtual memory or processor.

The invention also provides an apparatus including a nomogram. The apparatus including a nomogram may further comprise a storage mechanism, wherein the storage mechanism stores the nomogram; an input device that inputs the set of factors determined from a patient into the apparatus; and a display mechanism, wherein the display mechanism displays the quantitative probability of the risk of BPH progression. The storage mechanism may be random access memory, read-only memory, a disk, virtual memory, a database, and a processor. The input device may be a keypad, a keyboard, stored data, a touch screen, a voice activated system, a downloadable program, downloadable data, a digital interface, a hand-held device, or an infra-red signal device. The display mechanism may be a computer monitor, a cathode ray tub (CRT), a digital screen, a light-emitting diode (LED), a liquid crystal display (LCD), an X-ray, a compressed digitized image, a video image, or a hand-held device. The apparatus may further comprise a display that displays the quantitative probability of the risk of BPH progression, e.g., the display is separated from the processor such that the display receives the quantitative probability of the risk of BPH progression. The apparatus may further comprise a database, wherein the database stores the correlation of factors and is accessible by the processor. The apparatus may further comprise an input device that inputs the set of factors determined from the patient diagnosed as having BPH into the apparatus. The input device stores the set of factors in a storage mechanism that is accessible by the processor. The apparatus may further comprise a transmission medium for transmitting the selected set of factors. The transmission medium is coupled to the processor and the correlation of factors. The apparatus may further comprise a transmission medium for transmitting the set of factors determined from the patient diagnosed as having BPH, preferably the transmission medium is coupled to the processor and the correlation of factors. The processor may be a multi-purpose or a dedicated processor. The processor includes an object oriented program having libraries, said libraries storing said correlation of factors.

In addition to assisting the patient and physician in selecting an appropriate course of therapy, the nomograms of the present invention are also useful in clinical trials to identify patients appropriate for a trial, to quantify the expected benefit relative to baseline risk, to verify the effectiveness of randomization, to reduce the sample size requirements, and to facilitate comparisons across studies.

Exemplary Methods, Apparatus and Nomograms with 5β-Reductase Inhibitor Therapy

In addition to the various embodiments of the nomograms and method of using the nomograms discussed above, the present invention is also directed toward nomograms and methods of utilizing these nomograms to predict the probability of BPH progression with drug therapy, including a 5 alpha reductase inhibitor, such as dutasteride or finasteride, an alpha blocker, a novel medical therapy for BPH, or a combination thereof. Comparison of the probability generated with the use of above nomograms, which predicts the probability of BPH progression without drug therapy, with the probability generated from nomograms which predict the probability of BPH with drug therapy can lead to the probability of reduction of the risk of BPH with drug therapy, including a 5 alpha reductase inhibitor, such as dutasteride or finasteride, or an alpha blocker, or other medical therapy for BPH, or a combination thereof. This prognosis may be utilized, among other reasons, to determine the usefulness of drug therapy in a BPH patient.

Accordingly, further embodiments of the present invention include nomograms which incorporate drug therapy, specifically a 5 alpha reductase inhibitor, such as dutasteride or finasteride, an alpha blocker, or other drug therapy for BPH, or a combination thereof, to predict BPH progression with drug treatment. In one embodiment, drug therapy includes therapy with dutasteride, finasteride, placebo, or a combination thereof.

One embodiment of the invention is directed to a method to predict the risk of BPH progression, e.g., AUR and/or SI, in a patient with drug therapy, while another embodiment of the invention is directed to predicting the probability of progression of BPH, AUR and/or SI in a patient without drug therapy, both methods include detecting or determining a plurality of factors comprising in level of PSA, PV, Qmax, AUA-SI score, BII score, and/or prior use of alpha blockers along with drug therapy; and correlating the level, value or score of the plurality of factors comprising PSA level, PV, Qmax, AUA-SI score, BII score, and/or prior use of an alpha blocker(s) along with other drug therapy with the risk, or with the probability, of progression of BPH with therapy. In another embodiment, the factors include one more of the following factors: age, PSA level, AUA-SI score, BII score, Qmax, PV, PVR and/or BPSA level. In one embodiment, the factors include one more of the following factors: factors: age, ethnicity, PSA level, AUA-SI score, BII score, Qmax, PVR, proPSA level, intact non-complexed PSA level, JM-27 level, caveolin-1 level, caveolin-2 level, PV, prior use of an alpha blocker(s) and/or BPSA level. In one embodiment, prior alpha blocker use is not a factor.

In one embodiment, the correlating may be accomplished by computer. In one embodiment, the correlating includes accessing a memory storing the selected set of factors. In another embodiment, the correlating includes generating a functional representation and displaying the functional representation on a display. In one embodiment, the displaying includes transmitting the functional representation from a source. In one embodiment, the correlating is executed by a processor or a virtual computer program or interactive web site. In another embodiment, the method further comprises transmitting the quantitative probability of BPH progression. In yet another embodiment, the method further comprises inputting the identical set of factors for the patient within an input device. In another embodiment, the method further comprises storing any of the set of factors to a memory or to a database.

Another embodiment of the invention is directed to an apparatus for predicting the probability of a reduction of the risk of BPH progression in a BPH patient with drug therapy. The apparatus comprises a data input means, for input of test information comprising detecting or determining drug therapy along with one or more of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of an alpha blocker(s) and/or BPSA level, a processor, executing a software for analysis of drug therapy, along with the amount, level or score of one or more of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of an alpha blocker(s) and/or BPSA level; wherein the software analyzes the use of drug therapy, along with the amount, level or score of one or more of the following factors: age, ethnicity, PSA level, AUA-SI score, BII score, Qmax, PVR, proPSA level, intact non-complexed PSA level, JM-27 level, caveolin-1 level, caveolin-2 level, PV, prior use of an alpha blocker(s) and/or BPSA level, and provides the risk of BPH progression in the mammal. In one embodiment, prior alpha blocker use is not a factor. Comparison of the risk of BPH progression with therapy to that without therapy results in the prediction of the probability of a reduction of the risk of BPH progression in a BPH patient with therapy.

Another embodiment of the invention is directed to a nomogram. The nomogram may be generated with a Cox proportional hazards regression model (Cox 1972). Alternatively, the nomogram may be generated with a neural network model (Rumelhart et al. 1986). In another embodiment, the nomogram is generated with a recursive partitioning model (Breiman et al. 1984). In yet another embodiment, the nomogram is generated with support vector machine technology (Cristianni et al. 2000). Other models known to those skilled in the art may alternatively be used. In one embodiment, the invention includes the use of software that implements Cox regression models or support vector machines to BPH progression.

The nomogram may be the graphic representation of a probability that a BPH patient will experience a risk of BPH progression with therapy comprising a set of indicia on a solid support, the indicia comprising one or more factor lines including an age line, an ethnicity line, a PSA level line, an AUA-SI score line, a BII score line, a Qmax line, a PVR line, a proPSA level line, an intact non-complexed PSA level line, a JM-27 level line, a caveolin-1 level line, a caveolin-2 level line, a PV line, a drug therapy line, a prior use of an alpha blocker(s) line and/or a BPSA level line, a points line, a total points line and a predictor line, wherein the age line, ethnicity line, PSA level line, AUA-SI score line, BII score line, Qmax line, PVR line, proPSA level line, intact non-complexed PSA level line, JM-27 level line, caveolin-1 level line, caveolin-2 level line, PV line, drug therapy line, prior use of an alpha blocker(s) line and/or BPSA level line each have values on a scale which can be correlated with values on a scale on the points line, and wherein said total points line has values on a scale which may be correlated with values on a scale on the predictor line, such that the value of each of the points correlating with the patient's age, ethnicity, PSA level, AUA-SI score, BII score, Qmax, PVR, proPSA level, intact non-complexed PSA level, JM-27, caveolin-1, caveolin-2, PV, drug therapy, prior use of an alpha blocker(s) and/or BPSA level can be added together to yield a total points value, and the total points value can be correlated with the predictor line to predict the probability that a BPH patient will experience a risk of BPH progression with therapy. In one embodiment, prior alpha blocker use is not a factor. Comparison of this probability with that of the above nomogram for the prediction of risk of BPH without therapy results in the probability that BPH risk can be reduced with therapy. The solid support may assume any appropriate form such as, for example, a laminated card. Any other suitable representation, picture, depiction or exemplification may be used.

The nomogram may assume any form, such as a computer program, e.g., in a hand-held device, world-wide-web page, e.g., written in FLASH, or a card, such as a laminated card. Any other suitable representation, picture, depiction or exemplification may be used. The nomogram may comprise a graphic representation and/or may be stored in a database or memory, e.g., a random access memory, read-only memory, disk, virtual memory or processor.

The invention also provides an apparatus including a nomogram. The apparatus including a nomogram may further comprise a storage mechanism, wherein the storage mechanism stores the nomogram; an input device that inputs the set of factors determined from a patient into the apparatus; and a display mechanism, wherein the display mechanism displays the quantitative probability of the risk of BPH progression with 5 alpha reductase therapy. The storage mechanism may be random access memory, read-only memory, a disk, virtual memory, a database, and a processor. The input device may be a keypad, a keyboard, stored data, a touch screen, a voice activated system, a downloadable program, downloadable data, a digital interface, a hand-held device, or an infra-red signal device. The display mechanism may be a computer monitor, a cathode ray tub (CRT), a digital screen, a light-emitting diode (LED), a liquid crystal display (LCD), an X-ray, a compressed digitized image, a video image, or a hand-held device. The apparatus may further comprise a display that displays the quantitative probability of the risk of BPH progression with 5 alpha reductase therapy, e.g., the display is separated from the processor such that the display receives the quantitative probability of the risk of BPH progression with 5 alpha reductase therapy. The apparatus may further comprise a database, wherein the database stores the correlation of factors and is accessible by the processor. The apparatus may further comprise an input device that inputs the set of factors determined from the patient diagnosed as having BPH into the apparatus. The input device stores the set of factors in a storage mechanism that is accessible by the processor. The apparatus may further comprise a transmission medium for transmitting the selected set of factors. The transmission medium is coupled to the processor and the correlation of factors. The apparatus may further comprise a transmission medium for transmitting the set of factors determined from the patient diagnosed as having BPH, preferably the transmission medium is coupled to the processor and the correlation of factors. The processor may be a multi-purpose or a dedicated processor. The processor includes an object oriented program having libraries, said libraries storing said correlation of factors.

In addition to assisting the patient and physician in selecting an appropriate course of therapy, the nomograms of the present invention are also useful in clinical trials to identify patients appropriate for a trial, to quantify the expected benefit relative to baseline risk, to verify the effectiveness of randomization, to reduce the sample size requirements, and to facilitate comparisons across studies.

One embodiment of the invention is directed to a method for predicting reduction of the risk of BPH progression in a patient. According to the methods, apparatus and nomograms discussed in the above section, the risk of BPH progression in a patient without therapy can be predicted. By adding the factor “drug therapy” into the factors to be detected or determined, the risk of BPH progression with the start of drug therapy can be predicted. If the risk of BPH progression is lower with drug therapy included, then a reduction of the risk of BPH progression with drug therapy has been determined. This method can greatly aid a medical practitioner in the treatment of his/her patients.

Exemplary Methods, Apparatus and Nomograms with For Risk of BPH Progression and Development of Prostate Cancer

In addition to the various embodiments of the nomograms and method of using the nomograms discussed above, the present invention is also directed toward nomograms and methods of utilizing these nomograms to predict the probability of BPH progression and prostate cancer, with or without drug therapy, including a 5 alpha reductase inhibitor, such as dutasteride or finasteride, an alpha blocker, or other medical therapy for BPH or a combination thereof. The methods, apparatus and nomograms are described herein and are similar to those described above with the consideration of one or more of the following factors: age, ethnicity, PSA level, AUA-SI score, BII score, Qmax, PVR, proPSA level, intact non-complexed PSA level, JM-27, caveolin-1, caveolin-2, PV, prior use of alpha blockers, family history of prostate cancer, status of previous biopsies and/or BPSA level, to determine the risk of BPH progression and prostate cancer development. One or more of the following the factors may also be considered: the level or amount of VEGF, UPAR, UPA, sVCAM, TGF-β₁, IL6sR, IL6, and/or a Gleason score. The nomogram and methods of using the nomogram may also include drug therapy, including a 5 alpha reductase inhibitor, such as dutasteride or finasteride, an alpha blocker, a novel medical therapy for BPH or a combination thereof as a factor. In one embodiment, prior alpha blocker use is not a factor.

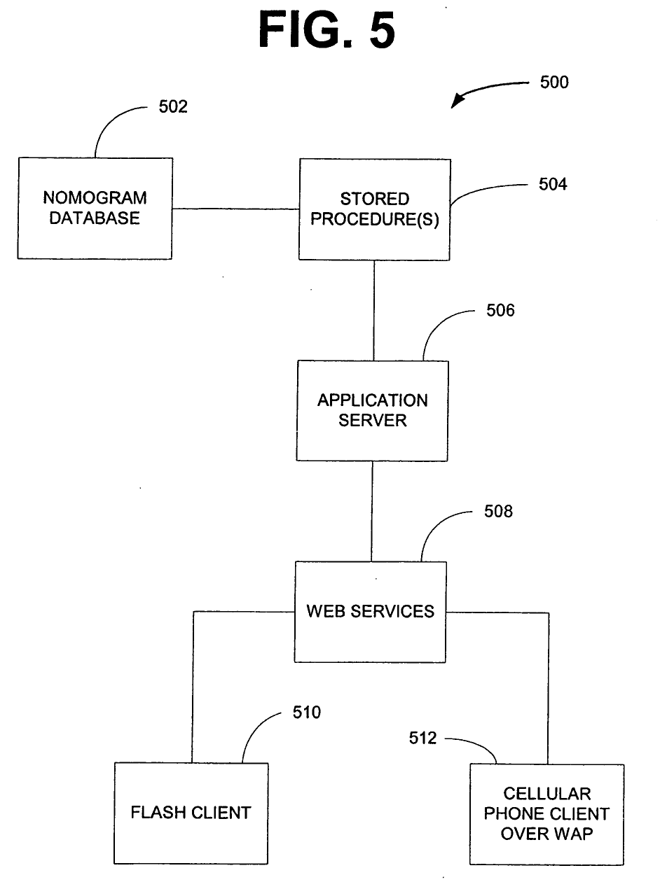

FIG. 5 illustrates an exemplary embodiment of a nomogram system architecture 500. The nomogram system architecture 500 provides centralized storage of nomograms. This provides administrators the ability to administer and implement nomogram modifications, additions, and deletions quickly and efficiently. Further, the centralized storage of nomograms reduces the amount of data that is stored on user systems utilizing the nomogram system architecture 500. Additionally, some embodiments include a single algorithm to compute nomogram results, which increases efficiency and accuracy in development of new nomograms. Further, this architecture streamlines deployment of nomograms and provides a mechanism to control nomogram access.

The nomogram system architecture 500 includes a nomogram database 502 that is accessible via one or more stored procedures 504. The nomogram system architecture 500 further includes an application server 506 to service request from and through a web services server 508 that includes services to communicate with one or more client types such as a Macromedia Flash client 510, a cellular phone client 512, or other client types (not illustrated).

The nomogram database 502 includes representations of nomograms to predict progression of various ailments including the progression of benign prostatic hyperplasia (BPH) as described above. The nomogram database 502, in various embodiments, is a relational database such as Microsoft SQL Server, a hierarchical database, a flat file arrangement of nomograms, or virtually any other arrangement of data that allows access to the data based on one or more other items of data, used as a key(s), included in the nomogram database 502.

The nomogram database 502 of the example embodiment illustrated in FIG. 5 is accessible to users of the nomogram system via stored procedures 504. The stored procedures 504 can be written in a proprietary language of the specific database of a particular embodiment, such as Stored Procedure Language (SPL) of Microsoft SQL Server. In other embodiments, the stored procedures 504 are written in another compiled or uncompiled programming or scripting language as necessary based on the requirements of the specific embodiment. The stored procedures 504 access data in the nomogram database 504 and can perform calculations on the data based on requests from the application server 506. The calculations can include virtually any type of calculation, such as averaging and interpolation of the data, necessary to predict the progression of various ailments for which nomograms exist in the nomogram database 502.

The application server 506 can be virtually any application server. In some embodiments, the application server 506 is based on the Microsoft .Net platform. In other embodiments, the application server operates using an open source application server platform such as Tomcat. The application server 506 operates to service transactions requiring nomogram database 502 access. The application server 506 receives transaction requests from clients over a network, such as the Internet or a mobile telephone network, encoded according to a protocol such as a web services protocol. The application server 506 communicates with clients to provide generic access controls to multiple clients via user interfaces operable on the clients. Communication between the application server 506 and the clients includes utilizing TCP/IP, COM, DCOM, XML, Simple Object Access Protocol (SOAP), Web Services Description Language (WSDL), and other related connection communication protocols and technologies that will be readily apparent to one of skill in the relevant art. In some embodiments, such as is illustrated in FIG. 5, the nomogram system architecture 500 includes a web services server 508 to handle communication and translation of web services transactions between the application server 506 and clients, such as the Flash client 510 and the cellular phone client 512.

In some embodiments, the application server includes a generic nomogram class. This class can be instantiated for each of the nomograms stored in the nomogram database. The class merely requires definition of the various components of a nomogram and the instantiated class can then perform the required database lookups and probability calculations necessary to predict progression of the ailment of the particular nomogram.

Clients, such as the Flash client 510 and the cellular phone client 512 communicate with the application server 506 over a network, such as the Internet or a mobile telephone network, to request and receive a client user interface. The client user interface is received over the network to display information, receive data, request data, and present request results to a client user.

In some embodiments, the client is a personal computer operatively connected to the Internet. The user interface of such a client, in some embodiments, is communicated to and operable on the client in a markup language, such as HTML. Such user interfaces are displayable on these clients in a web browser, such as a Microsoft's Internet Explorer. In other embodiments, the client is a mobile telephone that communicates with the application server according to the Wireless Application Protocol (WAP). Such mobile telephone embodiments include a user interface that receives input from a user, communicates that input to the application server 506, and receives and displays a predicted progression of various ailments based on data in the nomogram database 502.

In some embodiments, the nomogram database 502, the stored procedures 504, the application server 506, and the web services server 508 all reside on the same physical server. However, in various other embodiments, these components reside on two or more physical servers or other computers and are operably connected to service clients via a network, such as a system area network (SAN) or local area network (LAN), which is also connected to a wide area network (WAN), such as the Internet.

The invention will be further described by the following non-limiting examples.

### EXAMPLE 1

### Nomograms to Predict BPH Progression with or without Dutasteride Therapy

Benign prostatic hyperplasia (BPH) is a chronic and progressive condition associated with a significant risk of acute urinary retention (AUR) and need for surgical intervention (Emberton et al. 2002). A 60 year-old man has a 23% lifetime risk of AUR (Jacobsen et al. 1991)), whilst a man aged ≧60 years with an enlarged prostate and obstructive symptoms has a 39%, 20-year probability of undergoing BPH-related surgery (Arrighi et al. 1991).

Risk factors for progression to outcomes such as AUR and the need for surgery can be used to identify men at higher risk (Emberton et al. 2002), and can facilitate timely initiation of medical therapy with 5α-reductase inhibitors (5ARIs), which have demonstrable efficacy in reducing the risk of these outcomes (McConnell et al. 1998; Roehrborn et al. 2002). For example, baseline prostate volume (PV) and serum prostate-specific antigen (PSA) levels have been shown to be predictors of prostate growth, and an increased risk of AUR and BPH-related surgery (Roehrborn et al. 2002).

The use of single parameters to predict the risk of BPH progression or to determine the relative reduction potentially gained with 5ARI therapy is not optimal. Nomograms are prediction tools optimized for accuracy that utilize multiple parameters to predict specific outcomes. The objective of this study was to develop a prediction model, or nomogram, that would predict the probability that a man with BPH would experience AUR or require surgical intervention (SI) within two years, using data from the recently completed Phase III studies of dutasteride versus placebo in men with BPH (Roehrborn et al. 2002).

Methods

Data from three two-year multicenter, placebo-controlled, double-blind studies evaluating dutasteride 0.5 mg/day (n=2,167) or placebo (n=2,158) in male subjects with BPH were utilized to develop a nomogram. Subjects were at least 50 years of age, had a serum PSA>1.5 ng/mL and <10 ng/mL, had BPH diagnosed by medical history and physical examination that revealed an enlarged prostate (30 cc), and had BPH symptoms that were moderate to severe according to the American Urological Association Symptom Index (AUA-SI). Most of the 4,325 subjects randomly assigned to receive either dutasteride or placebo completed 2 years of treatment (70% and 67%, respectively).

Subjects were characterized at baseline by a number of parameters, including AUA symptom index (AUA-SI) score, BPH impact index (BII) score, prostate volume, prostate specific antigen (PSA) level, maximum urinary flow rate (Qₘₐₓ), and prior use of selective α₁ blockers. Cox proportional hazards regression was used to relate these baseline variables to the future probability of developing AUR or requiring surgical intervention within 2 years. The nomogram was internally validated with bootstrapping, a re-sampling technique, to assess its discrimination and calibration. Discrimination was quantified as the concordance index, which is rated from 0.5 to 1.00. Calibration was assessed visually, by plotting observed proportions against predicted probabilities, again using bootstrapping to reduce over-fit bias.

Results

In the phase III studies, dutasteride treatment resulted in a 57% reduction in the risk of AUR and a 48% reduction in the need for BPH-related surgery over the 24 month duration of the study. At endpoint, 6.8% of placebo-treated patients and 3.5% of dutasteride-treated patients had experienced AUR and/or surgical intervention, representing a 50% relative risk reduction in patients receiving dutasteride treatment.

The hazard ratios for predictors in the full multivariate model are shown in Table 1. The unit of change associated with the hazard ratios for continuous variables (baseline serum PSA, prostate volume, BII score, Qₘₐₓ, and AUA-SI) is provided in the first column. The other two predictors, selective α₁ blockers and randomization group (dutasteride versus placebo group), are dichotomous variables.

TABLE 1

| | Hazard Ratio | |
|---|---|---|
| Variable | (95% Cls) | p-value |
| AUA-SI | 1.17 (0.95, 1.45) | 0.141 |
| BII | 1.35 (1.08, 1.68) | 0.008 |
| Prior α-blockers | 1.58 (1.20, 2.09) | 0.001 |
| Prostate volume (cc) | 1.29 (1.15, 1.45) | 0.001 |
| PSA (ng/ml) | 1.35 (1.12, 1.62) | 0.002 |
| Qₘₐₓ (ml/sec) | 0.60 (0.50, 0.73) | 0.001 |
| Dutasteride therapy | 0.50 (0.37, 0.66) | 0.001 |

In multivariate analysis, baseline serum PSA, prostate volume, BII score, Qₘₐₓ and a prior requirement for selective α₁ blockers treatment were all predictors of BPH progression at the 5% level of significance. AUA-SI was not a significant predictor of progression.

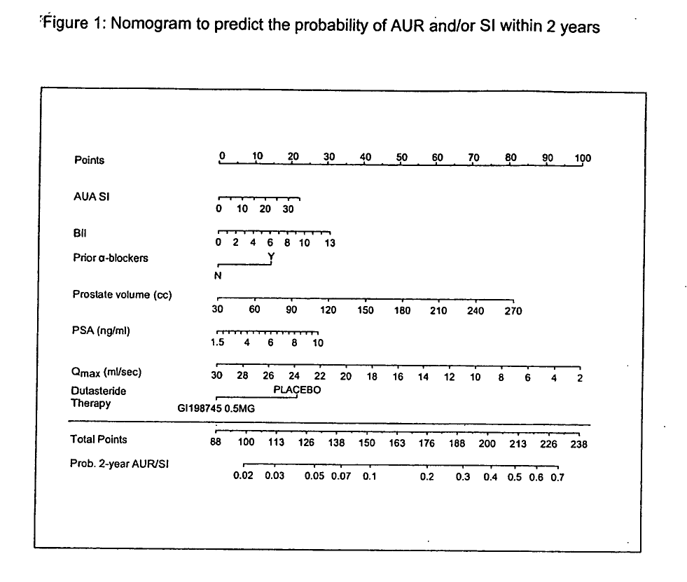

The nomogram appears in FIG. 1. The value for each variable is associated with a corresponding number of points, based on weighting from the Cox proportional hazards regression model. The total number of points for all variables is used to determine the probability of AUR and/or surgical intervention within 2 years. The nomogram was evaluated for its ability to determine a patient's risk of BPH progression, as measured by the area under the curve for censored data (i.e., the concordance index). This value represents the probability that when two patients are randomly selected (one with who experienced progression and one who did not within the same length of followup), the patient who progressed first had the worse prognosis as predicted by the nomogram. This measure can range from 0.5 (no better than the chance “flip of the coin”) to 1.0 (perfectability to discriminate). To derive an estimate of expected performance of the nomogram for new patients, bootstrapping was performed, a statistical method in which sampling, nomogram building, and nomogram evaluation are repeated a large number of times. Using this method, the nomogram was shown to discriminate well, with a bootstrap-corrected concordance index of 0.71 (p<0.001).

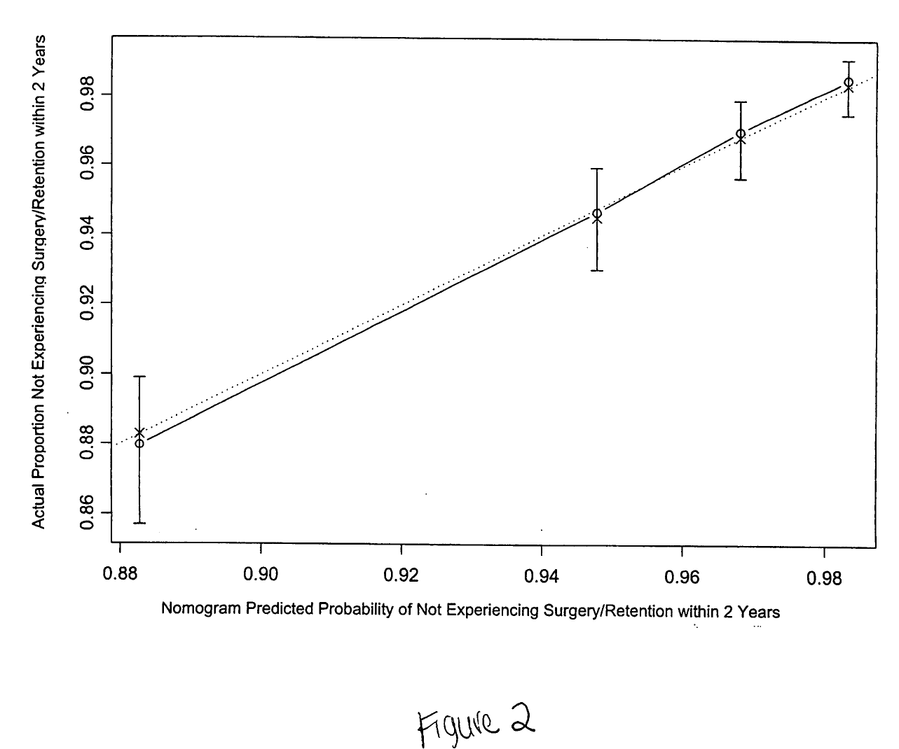

FIG. 2 illustrates how the predictions from the nomogram compare with actual outcomes for the entire cohort of patients. The x-axis is the prediction calculated with the use of the nomogram, and the y-axis is the actual risk of AUR/SI experienced by these patients. The solid line represents the performance of a perfectly accurate nomogram, in which predicted outcome perfectly corresponds to actual outcome. The nomogram's performance is plotted as a dashed line that connects the points corresponding to subcohorts (on the basis of predicted risk) within the dataset, with confidence intervals noted as well. Because the points lie relatively close to the solid line, and encompass the solid line well within the boundaries of the confidence intervals for each point, the predictions calculated with the nomogram approximate the actual outcomes.

Discussion

Despite the increasing number of published studies that have added to the general knowledge about the best candidate for selective α₁ blockers and 5-α—reductase inhibitors, physicians and patients have had few tools to help them translate this body of general knowledge into individualized, evidence-based recommendations or answer clinically important questions that they face on a daily basis. For BPH patients, these important clinical questions include:

1. What is the long-term risk of experiencing BPH progression in this patient?

2. Will this patient experience a significant reduction in BPH symptoms if medical therapy is initiated?

3. What would the reduction in risk of developing BPH progression be if I start the patient on a 5 α reductase inhibitor?

Unfortunately, physicians currently are not equipped to provide answers to these questions, that are tailored for individual patients. Until now, physicians have been encouraged to make clinically important decisions based on only one or just a few of the important parameters that affect the course of treatment for their patients. An example is the considerable confusion over the appropriate cut points to use in decisions regarding prostate cancer and BPH therapy. For BPH, physicians often agree that a 5ARI is appropriate for patients with a “large prostate”, but they often disagree as to whether this includes patients with prostate volumes>30 cc, >40 cc, or larger? Similarly, most urologists agree that higher PSA's are associated with a greater risk of finding prostate cancer on prostate biopsy, but disagree as to whether the correct cut point for prostate biopsy should be PSA>4, >2.5, or whether age-specific cut-points should be applied. Nomograms allow physicians to individualize these decisions, rather than applying a “one-size fits all” approach to medical decision-making.

Nomograms that incorporate diagnostic and clinical information can provide personalized, evidence-based answers to clinically important questions. A nomogram is a device or model that uses an algorithm or mathematical formula to predict the probability of an outcome, optimized for predictive accuracy. Nomograms, which allow continuous variables to remain continuous, thus maximizing their predictive power, provide complex predictions that are optimized for accuracy. They allow for the convergent use of all important data parameters, so that the most accurate prediction model can be built. Furthermore, nomograms can be continuously updated by building on prior knowledge rather than replacing it. Thus, novel markers, like BPSA, proteomics and genomics are evaluated by their ability to improve the overall accuracy of prediction models and are added to nomogram models when they provide significant improvement in the accuracy of predictions.

In order to generate a nomogram to predict BPH progression, key risk factors were assembled. For BPH, these risk factors were suggested through analyses of population-based and clinical trials databases. These risk factors have included higher age, more severe (obstructive) symptoms, lower Qₘₐₓ, greater prostate volume, a large endovesical lobe, and elevated serum PSA. While each of these individually is a risk factor for BPH progression, for individual patients, an increasing number and severity of these risk factors increases the absolute risk of BPH progression accordingly. Identifying patients at highest risk for BPH progression, while improving patient care decision-making at the individual patient level, also allows the selection of patients who would receive the greatest benefit from 5ARI/combination therapy.

With these benefits in mind, a nomogram was constructed to predict the risk of BPH progression using data from a phase III pivotal trial (ARIA 3001, 3002, 3003) used to establish the safety and efficacy of dutasteride prior to FDA approval. For each individual predictive parameter, points are awarded by drawing a perpendicular line to the point scale along the top of the nomogram. After all points are added, a perpendicular line is drawn from the bottom, total points scale to the line below, indicating the 2 year probability of a patient's developing retention or requiring BPH-related surgery within two years. Note that the use of dutasteride (versus placebo) leads to a total point score reduced by approximately 20 to 25 points, which translates to a 50% relative risk reduction across the entire range of total points for any patient. This nomogram was shown to have an accuracy of about 71%, better than the flip of coin (50%) but less than 100% perfect predictive accuracy. This research nomogram demonstrated that while the median risk of progression to a combined endpoint of AUR/surgery was only 6.8%, the maximum risk of progression in the most severely affected patients was 27% at two years, an absolute increase in the risk of progression of >20%. Thus, a 50% relative risk reduction over two years translates into about 13 to 14% absolute risk reduction over this very short time frame, with a much greater benefit likely experienced over time.

Careful examination of the linear relationships amongst the prediction parameters and between each parameter and the point scale yields important insights into the clinical importance of these parameters in predicting AUR/surgery. For example, the length of the axis for any one parameter along the point scale is a measure of the importance of that parameter within the overall prediction model. For this nomogram, therefore, it is clear that Qₘₐₓ and prostate volume are far more important than serum PSA level in predicting an AUR/surgery endpoint. This is an instructive lesson in the clinical differences between multivariable prediction models that utilize continuous variable predictors and the use of univariable predictors in clinical decision making.

Most of the significant predictive parameters evaluated in the present multivariable model have been previously recognized as important predictors of BPH progression (e.g., PSA and prostate volume). However, this study clearly demonstrated that the BII (HR 1.35, p-value 0.008), but not the AUA-SI (HR 1.17, p-value of 0.141), was a significant predictor of BPH progression in this study. Previous studies evaluating AUA-SI as a univariable predictor have suggested that this parameter was a significant predictor of future BPH-related surgery. In a multivariable model that included age, symptom severity, flow rate, and prostate volume, Jacobsen et al. showed that symptom severity was a significant predictor of medical and/or surgical treatment for BPH within the subsequent 6 year period in a cohort of 2,115 men, aged 40 to 79 years who were randomly selected from residents of Olmsted County, but no disease-specific QOL instrument, like the BII, was included in the model. Interestingly, recent analyses of the MTOPS data demonstrates a similar predictive power for the BII, but not the AUA-SI, in predicting future urinary retention and/or BPH-related surgery, suggesting that this instrument may be underutilized by physicians in their routine assessment and management of patients with BPH (data not shown).

Certain caveats exist with regard to the use of such a nomogram to predict similar outcomes in de novo patients. Since the nomogram was constructed using a population of patients restricted to those with a serum PSA level between 1.5 ng/mL and 10 ng/mL, de novo patients with a serum PSA level either below 1.5 ng/mL or above 10 ng/mL are likely not candidates for risk predictions using this tool.

Conclusions

Patients and physicians desire accurate knowledge regarding the risks and benefits of therapy when contemplating any new course of treatment for any disease. Because nomograms can provide the most accurate predictions for individual patients, we developed a nomogram to predict the risk of BPH progression after 2 years for patients considering dutasteride therapy for symptomatic BPH.

### EXAMPLE 2

### Nomograms to Predict the Risk of BPH Progression Using Data from the MTOPS Trial

A MTOPS (Medical Therapy of Prostatic Symptoms) based nomogram to predict BPH progression, including symptom progression at three and five years, and AUR (Acute Urinary Retention)/BPH Invasive Therapy Progression at three and five years, was developed from the data obtained from the MTOPS trial with the use of Cox proportional hazards modeling with splines to relax linear assumptions. A similar nomogram was constructed as demonstrated above in Example 1, which identified the following predictors at baseline that were included in the final nomogram: AUA-SI, BII index, prior use of alpha blockers, PSA level, prostate volume, Qmax, randomization group (dutasteride or placebo). As described herein below, the same variables listed in Example 1 at a minimum along with other predictors, e.g., age, PVR, and the like, that were significant predictors of BPH progression on univariable analysis of the MTOPS data performed to date, are candidate predictors for a MTOPS nomogram.

Materials and Methods

Patient Population

Medical Therapy of Prostatic Symptoms (MTOPS) is a clinical research study sponsored by the National Institutes of Health (NIH). The study tested whether the oral drugs finasteride (Proscar®) and doxazosin (Cardura®), alone or together, can further delay or prevent further prostate growth in men with BPH.

MTOPS is the largest and longest study to test whether the drugs can stop noncancerous prostate growth. Seventeen U.S. medical centers recruited 2931 men diagnosed with symptomatic BPH between December 1995 and March 1998. The study doctors continued to follow these men through November 2001 on a quarterly basis. In addition to the clinical progression of BPH, MTOPS included evaluations of prostate volume by ultrasound, prostate histopathobiology, quality of life and urodynamics. (Funding by NIDDK (UO1-DK-46472), 1992-2002; IND 43,564; http://www.bsc.gwu.edu/mtops/; McConnell et al., 2003).)

Statistical Analysis

Cox proportional hazards modeling with splines to relax linear assumptions to develop MTOPS nomograms that predict BPH progression using the data from the MTOPS trial.

Sample Size Calculations

The sample size required to develop nomogram models using baseline clinical data is based on the total number of degrees of freedom associated with the predictive parameters utilized within the nomogram model. Typically, ten “events”, or patients who reach the endpoint to be predicted, are required to adequately power a nomogram model (Concato et al. 1995). Continuous variables contain two degrees of freedom. The number of degrees of freedom for categorical variables contains one minus the number of categories. Tests for variable interaction increase the number of degrees of freedom as well. For the purposes described herein, the potential variables that might be included in a MTOPS base clinical nomogram are as follows:

TABLE 2

| PREDICTIVE VARIABLE | DEGREES OF FREEDOM |
|---|---|
| Age | 2 |
| AUA-SI | 2 |
| BII Index | 2 |
| PSA level | 2 |
| Qmax | 2 |
| PVR | 2 |
| Finasteride (fin) or placebo | 1 |
| Doxazosin (dox) or placebo | 1 |
| Interaction between fin and dox | 1 |
| Novel BPH Marker (e.g., BPSA) | 2 |
| TOTAL | 17 |

Therefore, for a full model, that included all of the above listed variables, an 5 unbiased cohort of patients that included a total of at least 170 events would be required to adequately power a nomogram.

For the entire MTOPS cohort, the summary of endpoint events is as follows:

TABLE 3

| EVENT TYPE | PLAC | DOX | FIN | COMB | ALL |
|---|---|---|---|---|---|
| AUA Rise | 100 | 59 | 74 | 41 | 274 |
| Retention | 18 | 13 | 6 | 4 | 41 |
| Incontinence | 8 | 11 | 9 | 3 | 31 |
| UTI/urosepsis | 2 | 2 | 0 | 1 | 5 |
| Creatinine Rise | 0 | 0 | 0 | 0 | 0 |
| Total | 128 | 85 | 89 | 49 | 351 |
| BPH Invasive Therapy | 40 | 41 | 15 | 14 | 110 |

For a nomogram model that predicted AUR/BPH Invasive Therapy, even utilizing the entire MTOPS cohort of patients, there are only 151 total events (41 AUR plus 110 BPH Surgery). Given that some variables may not be incorporated in a final nomogram model, e.g., PSA since BPSA or PVR may be a substitute, 151 events are adequate to create a sufficiently powered base nomogram model that excluded a putative novel BPH clinical marker, e.g., BPSA, and one that included such a novel marker as a predictive parameter. For prediction of an AUA symptom index rise, the large number of events (n=274) makes a full model sufficiently powered.

Dataset/Nomogram Generation

From the master MTOPS database, a dataset is generated with a complete set of pre-randomization clinical data including, but not limited to:
a. Baseline Age;
b. Ethnicity;
c. Baseline AUA-SI;
d. Baseline BII;
e. Baseline PSA;
f. Baseline Prostate Volume (PV);
g. Baseline Qmax;
h. Baseline PVR;
i. Dox randomization group (dox or placebo);
j. Fin randomization group (fin or placebo);
k. All data regarding discontinuation of coded medications with associated dates;
l. Prostate volume at each measured time point (one year, and at endpoint/end of study);
m. “Failure” should be coded as the first type experienced: AUA 4 pt rise, AUR, BPH invasive therapy, incontinence, infection, or prostate cancer. This permits the development of a model that predicts each of these endpoints (or some combination) recognizing that a patient may first fail by another method and thereby not experience the failure type of interest; and
n. Days to the first form of failure should be noted.

Two versions of the datasets are generated. The first version is intended to treat with regard to treatment group indicator. The second version of the dataset considers treatment (drug vs. placebo) to be a time varying covariate. This dataset consists of columns for “treatment”, “start time”, “stop time” and “failure”. When a patient changes treatment (either discontinuation of drug or switch to open label), another record in the database is created. His first record indicates start and stop times for first “treatment”, and the second record consists of a start time equal to the prior record's stop time. While the first version of the dataset (intent to treat) allows the model to predict the probability of failure for the patient who starts (or does not start) drug, the second form of the dataset yields a model that predicts the probability of failure should the patient maintain drug or never switch to it if on placebo.

Discussion

Development of a Nomogram to Predict AUR or BPH Related Surgery with or without Medical Therapy in Men with BPH Based on the MTOPS Trial Outcomes Data

Two methods are utilized to develop two sets of nomograms. In the first method, a competing risks model is developed to predict AUR/BPH surgery with the other trial endpoints (AUA-SI 4 point rise, incontinence, infection) treated as competing risks. An intent-to-treat method is utilized for patients, so that patients who switch to open label medication, or who stop medication, are analyzed according to their original randomization group. In the second method, a competing risk model with treatment (drug vs. placebo) considered to be a time varying covariate is utilized. The first method allows the prediction of AUR/BPH related surgery for patients who begin in a certain treatment group, and the second allows the prediction of AUR/BPH related surgery for patients who maintain within a certain treatment group.

Development of a Nomogram to Predict Symptom Progression with or without Medical Therapy in Men with BPH Based on the MTOPS Trial Outcomes Data

Nomograms to predict symptom progression, as defined in the MTOPS trial as a 4 point rise in the AUA-SI from baseline, based on the MTOPS trial cohort, are developed. Again, two methods are utilized to develop two sets of nomograms. In the first method, a competing risks model is developed to predict symptom progression with the other trial endpoints (AUR, incontinence, infection, and the secondary endpoint of BPH-related surgery) treated as competing risks. An intent-to-treat method is utilized for patients so that patients who switch to open label medication, or who stop medication will analyzed according to their original randomization group. In the second method, a competing risk model with treatment (drug vs. placebo) considered to be a time varying covariate is utilized. The first allows the prediction symptom progression for patients who begin in a certain treatment group, and the second allows the prediction of symptom progression for patients who maintain within a certain treatment group.

Development of a Nomogram to Predict Prostate Growth with or without Medical Therapy in Men with BPH Based on the MTOPS Trial Outcomes Data

Nomograms to predict future prostate growth based on the MTOPS trial cohort are developed. After further analyses to confirm that alpha blocker therapy is not effective against prostate growth, the MTOPS cohort is reduced from four treatment groups to two: the first includes patients randomized to placebo plus those randomized to doxazosin and the second includes patients randomized to finasteride plus those randomized to finasteride plus doxazosin. Treatment is considered a time varying covariate. This allows the prediction of future prostate growth for patients who stay on finasteride versus those who are not on finasteride.

Development of a Nomogram to Predict BPH Progression with or without Medical Therapy in Men with BPH Based on the MTOPS Trial Outcomes Data

Models that predict overall BPH progression by the addition of probabilities from prediction of AUR or SI and symptom progression, rather than by creating a more complex model (as demonstrated when both endpoints were considered together in models based on the data from Example 1 and that resulted in a model with a lower CI than models predicting these endpoints separately) are developed.

### EXAMPLE 3

### Development of a BPH Nomogram to Predict BPH Progression that Incorporates BPSA as a Predictor Using Data and Frozen Sera from the Merck-Sponsored Proscar Long-term Efficacy and Safety Study (PLESS)

In the past, prostate related work focused on the study of the molecular forms of PSA found in prostate tissue harvested at radical prostatectomy from three clinically important, yet different, areas of the prostate: non-cancerous peripheral zone, peripheral zone cancer, and benign transition zone of the prostate (Song et al. 1997; Slawin et al. 1998). Early studies focused on quantifying, using Western Blot analysis, the levels of free PSA, complexed PSA, and ACT present in these areas of the prostate, since it was hypothesized that the forms of PSA found in prostate tissue, which are present in milligram per milliliter quantities, and thus much easier to study, would reflect the character of PSA found in serum at nanogram per milliter quantities. Later, more sophisticated studies using affinity columns and hydrophobic interaction column chromatography, culminated in the discovery of “BPSA” (“benign” PSA), a novel form of free PSA associated with nodular hyperplasia of the transition zone (Mikolajczyk et al. 2000). These studies also demonstrated a clear association of truncated molecular forms of proPSA with the prostate peripheral zone, including prostate cancer (Mikolajczyk et al. 2000). More recent studies using serum assays specific for these various molecular forms of free PSA (fPSA) have demonstrated that the majority of fPSA in the blood is comprised of BPSA, truncated forms of proPSA, and an additional form of intact, yet inactive, PSA.

BPSA is predominantly clipped at amino-acid residues Lys145-146 and Lys 182-183 and is elevated in the transitional zone epithelium of prostates with nodular BPH. More recently, is has been shown that BPSA is also present in seminal plasma (Mikolajczyk et al. 2000). A dual monoclonal antibody assay for BPSA (detection limit of 0.06 ng/mL) has been evaluated in men with symptomatic BPH, in men without clinical BPH, and in healthy subjects. The median BPSA level in patients with symptomatic BPH was significantly higher than that in the patients without BPH symptoms. In the healthy control group, BPSA was almost undetectable (Linton et al. 2003).

While total PSA has been established as the best currently available serum marker for BPH, its lack of specificity in predicting clinically important outcomes, and limited utility as a univariate predictor of these outcomes, remains a concern. Because it is now clear that serum total PSA is a heterogeneous mixture of multiple molecular forms of PSA with different origins and different clinical properties, serum levels of disease specific PSA forms, e.g., BPSA for BPH, comprising only a portion of measured serum total PSA, will, like levels of serum total PSA, not only predict total prostate and TZ volume, but also predict BPH progression in untreated patients, predict future prostate growth, and predict response to therapy, albeit with better sensitivity and specificity. Furthermore, BPSA will require less stratification of the test population, e.g., by age and biopsy status, making it more useful clinically.

Statistical Analysis

The sample size required to develop nomogram models using baseline clinical data is based on the total number of degrees of freedom associated with the predictive parameters utilized within the nomogram model. Typically, ten “events”, or patients who reach the endpoint to be predicted, are required to adequately power a nomogram model (Concato et al. 1995). Continuous variables contain two degrees of freedom. The number of degrees of freedom for categorical variables contains one minus the number of categories. Tests for variable interaction increase the number of degrees of freedom as well. With respect to this Example, the potential variables that might be included in an MTOPS base clinical nomogram are as follows:

TABLE 4

| PREDICTIVE VARIABLE | DEGREES OF FREEDOM |
|---|---|
| Age | 2 |
| AUA-SI | 2 |
| BII Index | 2 |
| PSA level | 2 |
| Qmax | 2 |
| PVR | 2 |
| Finasteride or placebo | 1 |
| Novel BPH Marker (e.g., BPSA) | 2 |
| TOTAL | 15 |

Therefore, for a full model, that included all of the above listed variables, an unbiased cohort of patients that included a total of at least 150 events would be required to adequately power a nomogram.

In the PLESS study, the overall incidence of AUR was 7% with placebo and 4% with finasteride (spontaneous AUR 4% with placebo and 1% with finasteride; precipitated AUR 3% with placebo and 2% with finasteride) and of BPH-related surgery, 10% in men taking placebo and 5% in men taking finasteride. Because of the small number of events, the entire cohort of baseline serum samples will be assayed for BPSA.

Sample Size

4788 at baseline and month 48.

Data Analysis

The predictive accuracy of base nomograms is compared with that of nomograms including baseline levels of BPSA by calculating the concordance index (CI) of each separate model. An improved performance and accuracy of nomogram models, as evidenced by a higher CI, that include BPSA levels as a predictive parameter, demonstrate that BPSA is a clinically important new marker for BPH.

Discussion

An example of a baseline nomogram that can predict BPH progression using standard clinical predictors is presented in Example 1 and FIG. 1, in which the following predictors were identified at baseline that were included in the final nomogram: AUA-SI, BII index, prior use of alpha blockers, PSA level, prostate volume, Qmax, and randomization group (dutasteride or placebo) (Kattan et al. 2003).

A similar nomogram was developed using data from the Merck-sponsored PLESS study. In this double-blind, randomized, placebo-controlled trial, 3040 men with moderate-to-severe urinary symptoms and enlarged prostate glands were randomized to receive either 5 mg of finasteride or placebo for four years. Symptom scores (on a scale of 1 to 34), urinary flow rates, and the occurrence of outcome events were assessed every four months in 3016 men. Prostate volume was measured in a subgroup of the men. Complete data on outcomes are available for 2760 men and frozen sera was archived at baseline, at one year, and at end of study.

BPSA levels in frozen, archived serum specimens from patients randomized to Merck's PLESS study at baseline, at one year, and at end of the study are measured. Baseline nomogram models to predict BPH progression and those that include BPSA levels at baseline and follow-up are developed. This nomogram is able to predict the probability that a man with BPH will experience acute urinary retention (AUR) or require surgical intervention (SI) within four years with or without Proscar® therapy. BPSA, if a clinically important marker for BPH, can improve performance and accuracy of nomogram models that include BPSA levels.

### EXAMPLE 4

### Development of a BPH Nomogram to Predict BPH Progression that Incorporates BPSA as a Predictor Using Data and Frozen Sera from the MTOPS

With respect to this Example, the potential variables that might be included in an MTOPS base clinical nomogram are as follows:

TABLE 5

| PREDICTIVE VARIABLE | DEGREES OF FREEDOM |
|---|---|
| Age | 2 |
| AUA-SI | 2 |
| BII Index | 2 |
| PSA level | 2 |
| Qmax | 2 |
| PVR | 2 |
| Finasteride or placebo | 1 |
| Doxazosin or placebo | 1 |
| Interaction between fin and dox | 1 |
| Novel BPH Marker (e.g., BPSA) | 2 |
| TOTAL | 17 |

Therefore, for a fill model, that included all of the above listed variables, an unbiased cohort of patients that included a total of at least 170 events would be required to adequately power a nomogram.

For the entire MTOPS cohort, the summary of endpoint events is as follows:

TABLE 6

| EVENT TYPE | PLAC | DOX | FIN | COMB | ALL |
|---|---|---|---|---|---|
| AUA Rise | 100 | 59 | 74 | 41 | 274 |
| Retention | 18 | 13 | 6 | 4 | 41 |
| Incontinence | 8 | 11 | 9 | 3 | 31 |
| UTI/urosepsis | 2 | 2 | 0 | 1 | 5 |
| Creatinine Rise | 0 | 0 | 0 | 0 | 0 |
| Total | 128 | 85 | 89 | 49 | 351 |
| BPH Invasive Therapy | 40 | 41 | 15 | 14 | 110 |

For a nomogram model that predicted AUR/BPH invasive therapy, even utilizing the entire MTOPS cohort of patients, there are only 151 total events (41 AUR plus 110 BPH Surgery). Given that some variables may not be incorporated in a final nomogram model, e.g., PSA, since BPSA or PVR may be a substitute, 151 events should suffice to create an adequately powered base nomogram model that excluded a putative novel BPH clinical marker, e.g., BPSA, and one that included such a novel marker as a predictive parameter. For prediction of an AUA symptom index rise, the large number of events (n=274) makes a full model sufficiently powered.

Sample Size

There are approximately 3047 baseline serum samples.

Data Analysis

The predictive accuracy of base nomograms is compared with that of nomograms including baseline levels of BPSA by calculating the concordance index (CI) of each separate model. If BPSA represents a clinically important new marker for BPH, improved performance and accuracy of nomogram models, as evidenced by a higher CI, that include BPSA levels as a predictive parameter, should be seen.

Discussion

Herein described is the development of a nomogram similar to that shown in Examples 1 and 3 using data from the MTOPS study. In this double-blind, randomized, placebo-controlled trial, 3047 men with moderate-to-severe urinary symptoms and enlarged prostate glands were randomized to receive either 1) doxazosin+placebo; 2) finasteride+placebo; 3) doxazosin+finasteride; or 4) placebo+placebo for a minimum of four and a maximum of six years, with an average follow-up of five years. Complete data on outcomes were available for 3047 men and frozen sera was archived at baseline.

BPSA levels in frozen, archived serum specimens from patients randomized to the MTOPS study at baseline are measured. Baseline nomogram models to predict BPH progression and those that include BPSA levels at baseline and follow-up are developed. This nomogram is able to predict the probability that a man with BPH will experience AUR or require SI within four years with or without Proscar® therapy. BPSA, if a clinically important marker for BPH, may improve performance and accuracy of nomogram models that include BPSA levels.

### EXAMPLE 5

### Development of a BPH Nomogram to Predict BPH Progression

A nomogram comprising one or more or all of the datasets obtained from each of Examples 1-5 to predict the progression of BPH, with or without drug therapy, including a 5 alpha reductase inhibitor, such as dutasteride or finasteride, an alpha blocker, a novel medical therapy for BPH or a combination thereof, and optionally other datasets useful to predict BPH progression, is generated.

### EXAMPLE 6

### Development of a BPH Nomogram to Predict BPH Progression and Prostate Cancer Development

A prostate health nomogram is generated which requires a range of input parameters for an individual patient and outputs two predictions in the circumstance that the patient does not start drug therapy: 1) the risk of developing prostate cancer and 2) the risk of developing progression of BPH. These risk predictions would also be determined for a BPH patient who then elects to start therapy. The factors/predictors used would be weighted differently depending on whether the model was predicting development of prostate cancer or BPH progression.

The predictors for the risk of BPH progression and the risk of prostate cancer development have some overlap; however, some are particular to one or the other. For example, PSA and age are predictors of both, but AUA symptom score is a predictor of BPH progression, but not prostate cancer, and family history of prostate cancer is a predictor of prostate cancer development but not BPH progression.

Examples of nomograms to predict BPH progression with and without drug therapy are given above, particularly in Examples 1-5. A nomogram to predict prostate cancer development includes one or more of the following factors: PSA, age, race, ethnicity, family history of prostate cancer, status of previous biopsies (number of biopsies, number of cores, HGPIN etc.). An example of such a nomogram can be found in Lopez-Corona et al. (2003), which is specifically incorporated by reference herein. A nomogram for the prediction of prostate cancer development and/or progression may also include one or more of the following factors: the amount or level of VEGF, UPAR, UPA, sVCAM, TGF-β₁, IL6sR, IL6, and/or a Gleason score. To predict the risk of development of prostate cancer and/or progression with the start of drug therapy, drug therapy, including a 5 alpha reductase inhibitor, such as dutasteride or finasteride, an alpha blocker, a novel medical therapy for BPH or a combination thereof, should be added to the list of factors to consider.

The factors for both nomograms are combined into one nomogram, for example, the data could be entered as a single entry web page (like that found at www.drslawin.com under nomograms), and then two different predictions, one for BPH progression and one for development of cancer, using two separate mathematical models (with each mathematical model having some of the same data/factors received from the patient in common) would be calculated and the prediction of patient outcome is then determined, e.g., predictions in the circumstance that the patient does or does not start 5 alpha reductase inhibitor therapy: 1) the risk of developing prostate cancer and 2) the risk of developing progression of BPH.

### EXAMPLE 7

### Serum BPSA Outperforms both Total PSA and Free PSA as a Predictor of Prostate Enlargement in Men without Prostate Cancer

Prostate volume is a key predictor of both progression and response to 5∀-reductase inhibitor therapy in patients with BPH. Prostate volume has been shown to predict acute urinary retention in both population-based studies and BPH medical therapy trials (Jacobsen et al. 1997; Roehrborn et al. 1999)

The relationship between the log of the serum PSA concentration and the log of the prostate volume, in patients without prostate cancer, is linear and dependent on age (Roehrborn et al. 1999). For any given serum PSA concentration, older patients have larger prostate volumes and a steeper rate of increase in prostate volume with rising serum PSA concentration. Therefore, it is not surprising that serum PSA is also a predictor of both outcome and response to 5∀-reductase inhibitor therapy in patients with BPH.

The following experiments were carried out to determine whether the serum concentration of BPSA, a distinct form of free prostate specific antigen (PSA) enriched in the nodular TZ (TZ) tissue of benign prostate hyperplasia (BPH), can predict TZ volume and diagnose BPH-associated prostate enlargement in patients without prostate cancer (Jacobsen et al. 1997; Roehrborn et al. 1999; Roehrborn et al. 1999).

Although free PSA has been extensively studied as a tool in the screening for prostate cancer, its relationship to prostate volume has received little attention. A handful of studies have shown a positive correlation between percent free PSA (% fPSA) and prostate volume in patients with prostate cancer (Stephan et al. 1997; Haese et al. 1997; Mettlin et al. 1999). However, in patients without prostate cancer, this correlation has been found to be either very weak or absent (Haese et al. 1997; Mettlin et al. 1999). In contrast, the absolute value of free PSA, not in a ratio to total PSA, was found in one study to have a log-linear relationship to prostate volume in patients without prostate cancer (Morote et al. 2000).

Autopsy studies suggest that age-related increases in prostate volume occur via two distinct processes: enlargement of BPH nodules and diffuse enlargement of the TZ (Maru et al. 2003; Berry et al. 1984). The presence of nodular BPH introduces significant variability in TZ weight, reducing the ability of age to predict prostate size. This suggests that a serum marker that correlates with the presence of TZ nodules may be the best predictor of both prostate size and growth potential.

A clipped form of free PSA, termed BPSA, was recently identified at levels 3 to 4 times higher in the nodular hyperplastic TZ tissue from patients with BPH than in normal TZ tissue from patients without BPH or from peripheral zone tissue (Mikolajczyk et al. 2000). Purified BPSA has a distinctive cleavage between lysine 182 and serine 183 that results in unique immunoreactivity (Wang et al. 2000). BPSA was recently shown to be elevated in patients with BPH (Linton et al. 2003). Because free PSA is composed of multiple distinct molecular forms of PSA that can originate from cancer, benign peripheral and TZ tissues, and BPH-associated nodular hyperplastic TZ tissue, BPSA could outperform both total and free PSA as a predictor of prostate enlargement. It was found, as described herein, that BPSA correlates better with TZ volume than does PSA and that it can predict clinically significant prostate enlargement better than PSA or free PSA.

Materials and Methods

As part of an institutional review board-approved study, 261 serum samples were prospectively collected from men who underwent a transrectal ultrasound (TRUS) (79 patients) or ≧10-core TRUS-guided biopsy (182 patients) at the Scott Department of Urology. Men who had undergone an ablative procedure (i.e., transurethral resection of the prostate) within 10 years or had received anti-androgen therapy within 6 months of the time the serum was collected were excluded from the study. Results of International Prostate Symptom Score (IPSS) questionnaires and medical and surgical history were obtained by chart review. The study population consisted of 91 consecutive patients who underwent a ≧10-core TRUS biopsy and were found to be free of prostate cancer.

PSA and free PSA tests were carried out using the Hybritech Tandem-MP assays (Beckman Coulter, Inc., San Diego, Calif.). Serum BPSA determinations were carried out with an immunoassay developed at Beckman Coulter, Inc. as described in Linton et al. (2003). All PSA, free PSA, and BPSA measurements were performed on serum samples collected within 6 months of the biopsy date. No serum sample was collected within the first 6 weeks after a biopsy. Samples were sent to an outside facility at 4° C. for measurement of PSA and free PSA, and then shipped frozen either to Beckman Coulter, Inc., or to the Baylor Prostate Center where they were thawed and assayed for BPSA.

Total prostate and TZ volumes were determined by TRUS using the prolate ellipsoid formula. The 12-core biopsy scheme consisted of sextant biopsies plus laterally directed biopsies at the apex, middle, and base. In the 10-core biopsy scheme, unlike the 12-core scheme, the sextant biopsies at the mid prostate were not performed. Additional TZ and lesion-directed biopsies were carried out at the discretion of the attending urologist.

Receiver-operator characteristics (ROC) curve, linear, and binary logistic regression analyses were carried out with SPSS 10.0 (SPSS, Inc., Chicago, Ill.). Specificities and cut-off values were derived from the ROC curves for each test.

Results

Of the 261 men enrolled in the study, 182 underwent a TRUS-guided biopsy. Of these, 91 had a negative biopsy consisting of at least 10 cores and were, therefore, categorized as free of prostate cancer. IPSS scores were obtained in 73 of the 91 (80%) biopsy-negative patients in the study, 9 of whom did not respond to the quality-of-life question.

The median age, prostate volume, and TZ volume were 64, 57 cc, and 31 cc respectively. The median PSA, free PSA, BPSA, and % fPSA were 4.9 ng/ml, 0.70 ng/ml, 0.22 ng/ml and 15.6 ng/ml, respectively (Table 7). The free PSA concentration was higher than the BPSA concentration in each individual patient. The median difference between free PSA and BPSA was 0.49 ng/ml. BPSA comprised, on average, 32% of free PSA.

TABLE 7
Characteristics of the patient population

| | Mean | Median | Range | N |
|---|---|---|---|---|
| Age | 63 | 64 | 42-85 | 91 |
| PSA (ng/ml) | 5.8 | 4.9 | 0.9-20.9 | 91 |
| % fPSA | 17.3 | 15.6 | 3.8-44.4 | 91 |
| Free PSA (ng/ml) | 0.95 | 0.70 | 0.10-5.70 | 91 |
| BPSA (ng/ml) | 0.32 | 0.22 | 0.02-1.84 | 91 |
| Free PSA-BPSA (ng/ml) | 0.63 | 0.49 | 0.07-5.50 | 91 |
| BPSA/Free PSA | 0.32 | 0.32 | 0.03-0.71 | 91 |
| Prostate Volume (cc) | 65 | 57 | 21-259 | 91 |
| TZ Vol (cc) | 39 | 31 | 6-185 | 91 |
| IPSS | 12 | 10 | 1-34 | 73 |
| IPSS QOL | 2 | 2 | 0-6 | 64 |

Key:
% fPSA= percent free PSA fraction,
IPSS= International Prostate Symptom Score,
IPSS QOL= quality of life question of IPSS

Correlation with Prostate Volume and Quality of Life

PSA, BPSA, and free PSA were all significantly correlated with prostate volume, TZ volume, and age. Serum levels of all three showed a trend toward a stronger correlation with TZ volume than with total prostate volume. Although statistically significant, the correlation coefficient between age and PSA was lower than that between age and either BPSA or free PSA. % fPSA failed to correlate significantly with TZ volume, and only weakly correlated with total prostate volume and age (Table 8).

TABLE 8

| | | | | Free | Free PSA- | |
|---|---|---|---|---|---|---|
| | | PSA | BPSA | PSA | BPSA | % fPSA |
| Prostate | Corr* | 0.52 | 0.65 | 0.63 | 0.48 | 0.23 |
| Volume | Sig** | 0.000 | 0.000 | 0.000 | 0.000 | 0.031 |
| TZ Volume | Corr | 0.55 | 0.67 | 0.64 | 0.48 | 0.18 |
| | Sig | 0.000 | 0.000 | 0.000 | 0.000 | 0.080 |
| Age | Corr | 0.24 | 0.38 | 0.40 | 0.37 | 0.26 |
| | Sig | 0.020 | 0.000 | 0.000 | 0.000 | 0.012 |
| IPSS | Corr | 0.16 | 0.16 | 0.13 | 0.08 | −0.06 |
| | Sig | 0.174 | 0.179 | 0.273 | 0.503 | 0.591 |
| IPSS QOL | Corr | 0.25 | 0.25 | 0.11 | 0.01 | −0.20 |
| | Sig | 0.046 | 0.046 | 0.368 | 0.959 | 0.113 |
| IPSS Question | Corr | 0.22 | 0.28 | 0.21 | 0.12 | −0.03 |
| 5 | Sig | 0.061 | 0.017 | 0.080 | 0.333 | 0.830 |
| (weak stream) | | | | | | |
| IPSS Question | Corr | 0.29 | 0.22 | 0.19 | 0.14 | −0.09 |
| 7 | Sig | 0.012 | 0.057 | 0.104 | 0.238 | 0.455 |
| (nocturia) | | | | | | |

Key:
% fPSA = percent free PSA fraction,
IPSS = International Prostate Symptom Score,
IPSS QOL = quality of life question of IPSS,
bold = statistically significant to the 95% level
*Spearman's rho correlation coefficient
**Significance level, 2-tailed

Both BPSA and free PSA had a stronger correlation with total prostate volume and TZ volume than did PSA. Subtracting BPSA from free PSA reduced the correlation of free PSA with total and TZ volume to a value lower than that of PSA alone (Table 8). This suggests that a significant portion of the correlation between free PSA and prostate volume is due to the BPSA fraction of free PSA.

There was a statistically significant positive correlation between IPSS QOL responses and both PSA and BPSA but not free PSA. When individual questions of the IPSS questionnaire were analyzed independently, a significant positive correlation existed between BPSA and the response to question number 5 relating to the degree of weakness of the urinary stream. Only PSA correlated with the responses to question 7 relating to degree of nocturia (Table 8).

Prediction of Transition Zone Volume

Using linear regression models, it was found that BPSA and free PSA have a log-linear relation to prostate volume and TZ volume. Unlike that of PSA, however, the relation of BPSA and free PSA to total prostate and TZ volumes is independent of age. These findings are demonstrated by including age and either logPSA concentration, logBPSA concentration, or logfree PSA concentration in separate linear regression models that predict the log of the TZ volume. Age approached significance as a predictor of TZ volume in the PSA-based model (P=0.072), but not in either the BPSA (P=0.709) or the free PSA-based (P=0.595) models (Table 9).

TABLE 9

| Variables in Model | Sig* | Adjusted R-Square |
|---|---|---|
| Log PSA | 0.000⁺ | 0.316 |
| Age | 0.072 | |
| Log BPSA | 0.000⁺ | 0.420 |
| Age | 0.709 | |
| Log free PSA | 0.000⁺ | 0.413 |
| Age | 0.595 | |
| Log PSA | 0.888 | 0.451 |
| Age | 0.928 | |
| Log BPSA | 0.010⁺ | |
| Log free PSA | 0.025⁺ | |

*Statistical significance of the contribution by each variable to the linear predictive model
⁺Statistically significant to the 95% level

Both BPSA and free PSA-based linear regression models outperformed a PSA and age-based model. The PSA and age-based model explained only 30% of the variation in TZ volume, whereas a BPSA-based model explained 36% and a free PSA-based model explained 37% of the variation in TZ volume. When age, logPSA, logBPSA, and logfree PSA were included in one linear regression model, only BPSA and free PSA remained independent predictors of TZ volume. This finding was confirmed by stepwise linear regression analysis.

Diagnostic Utility of BPSA In order to determine whether any of the three serum tests can provide clinically useful prediction of TZ volume, ROC curves were plotted for each of the three serum tests and the specificity was calculated at 95, 90, 85, and 80% sensitivity for three different TZ sizes. The specificity of BPSA for the prediction of TZ enlargement at all sensitivity levels was better than that of PSA. Only BPSA demonstrated a statistically significant (P<0.05) difference in the area under the curve (AUC) when compared with the AUC of PSA for TZ volume>30 cc. Although not statistically significant, there was a trend towards a larger AUC for BPSA as compared with free PSA for the prediction of TZ volumes>20, 30 or 40 cc (Table 10).

The ability of PSA, free PSA, and BPSA to predict clinically significant prostate enlargement was further examined using binary logistic regression analysis. When the concentration of all three markers was included in separate binary logistic regression models for each TZ volume, only BPSA provided independent predictive value in each of the three models (Table 9). This further confirms the ability of BPSA to outperform both PSA and free PSA for the prediction of TZ enlargement.

Discussion

BPSA was discovered as a distinct form of free PSA abundant in nodular BPH TZ tissue. Nevertheless, the relationship between serum levels of BPSA and parameters of BPH, such as prostate volume, has not yet been thoroughly explored. A number of variables, including rates of release into the circulation, rate of clearance, and stability in serum, among many others, likely affect the steady state concentration of BPSA in the bloodstream of patients. This study is the first to clearly demonstrate that serum BPSA concentration can predict TZ volume and diagnose prostate enlargement in patients without prostate cancer.

Unlike PSA, which has been shown in large studies to have an age dependent log-linear relationship to prostate volume, both free PSA and BPSA in our study, predicted TZ volume independently of age. Only one previously published study directly evaluated the relationship between free PSA and prostate volume (Morote et al. 2000). In agreement with that study, it was demonstrated herein that free PSA has a log-linear relationship with prostate volume and that free PSA predicts prostate volume better than PSA in patients without prostate cancer.

Free PSA is composed of the various proPSA isoforms (i.e., −2, −4, and −7 proPSA), intact free PSA, and BPSA (Mikolajczyk et al. 2000; Mikolajczyk et al. 1997; Mikolajczyk et al. 2001; Mikolajczyk et al. 2000; Mikolajczyk et al. 2000). ProPSA isoforms of free PSA are enriched in peripheral zone tissue and in serum of patients with prostate cancer, but are also found in non-cancer serum (Mikolajczyk et al. 1997; Mikolajczyk et al. 2001; Mikolajczyk et al. 2000; Mikolajczyk et al. 2000; Sokoll et al. 2003). BPSA comprised approximately 30% of the free PSA in this cohort of biopsy negative patients. When BPSA was substrated from free PSA, its correlation to prostate volume decreased to below that of PSA. Furthermore, there was a trend towards the correlation with prostate volume becoming dependent on age once BPSA was subtracted from free PSA (data not shown). Therefore, the ability of free PSA to predict TZ volume better than PSA appears to be significantly influenced by the contribution of BPSA.

TABLE 10
Diagnostic Utility of BPSA

| | | PSA | | BPSA | | free PSA | |
|---|---|---|---|---|---|---|---|
| TZ Vol | | Cut-off | | Cut-off | | Cut-off | |
| (cc) | Sensitivity (%) | (ng/ml) | Specificity | (ng/ml) | Specificity | (ng/ml) | Specificity |
| >40 | 95 | 1.9 | 11.9 | 0.15 | 45.8 | 0.46 | 41.0 |
| (N = 91) | 90 | 2.8 | 26.8 | 0.21 | 67.8 | 0.54 | 52.3 |
| | 85 | 3.1 | 32.2 | 0.22 | 85.4 | 0.59 | 56.7 |
| | 80 | 4.3 | 54.2 | 0.24 | 76.3 | 0.67 | 64.6 |
| | AUC | 0.758 | | 0.850 | | 0.803 | |
| | Regression** | P = 0.610 | | P = 0.009 | | P = 0.425 | |
| >30 | 95 | 1.9 | 13.6 | 0.10 | 43.2 | 0.42 | 41.9 |
| (N = 91) | 90 | 2.9 | 33.1 | 0.18 | 68.2 | 0.49 | 56.3 |
| | 85 | 3.7 | 50.0 | 0.19 | 72.7 | 0.55 | 63.8 |
| | 80 | 4.0 | 53.8 | 0.20 | 73.6 | 0.61 | 68.2 |
| | AUC | 0.749* | | 0.844* | | 0.799 | |
| | Regression | P = 0.888 | | P = 0.009 | | P = 0.443 | |
| >20 | 95 | 2.0 | 28.0 | 0.08 | 44.0 | 0.27 | 27.8 |
| (N = 91) | 90 | 2.9 | 52.0 | 0.10 | 56.0 | 0.42 | 59.4 |
| | 85 | 3.1 | 52.0 | 0.14 | 68.0 | 0.46 | 65.9 |
| | 80 | 3.8 | 68.0 | 0.17 | 76.0 | 0.50 | 72.0 |

| | | | |
|---|---|---|---|
| AUC | 0.808 | 0.868 | 0.855 |
| Regression | P = 0.667 | P = 0.024 | P = 0.387 |

Key:
TZ Vol = TZ volume,
AUC = area under the ROC curve
*statistically significant difference over entire curve at the 95% level
**statistical significance of the contribution by each variable to the binary logistic regression model

Both ROC and binary logistic regression analyses confirm that BPSA outperforms both PSA and free PSA in its ability to diagnose clinically significant prostate enlargement. Because the degree of prostate enlargement that results in BPH progression as measured by IPSS score, AUR, or need for surgery varies from study to study, the ability of BPSA to predict a relatively wide range of prostate enlargement, namely TZ volumes of >20 cc, >30 cc, and >40 cc that correspond to total prostate volumes of roughly 40, 57, and 67 cc was evaluated. For this range of prostate sizes, BPSA outperformed both PSA and free PSA to a statistically significant degree as measured by binary logistic regression analyses.

Finding a statistically significant correlation between IPSS QOL responses and both PSA and BPSA but not free PSA was notable, given the limited size of this study. Taken together with the significantly more accurate prediction of TZ volume over PSA, the data confirm the potential of BPSA as a new predictor of outcomes and/or response to therapy in BPH patients.

In summary, BPSA and free PSA showed stronger correlations with both age (BPSA=0.38, free PSA=0.40, PSA=0.24) and TZ volume (BPSA=0.67, free PSA=0.64, PSA=0.55) than did PSA. Percent free PSA had no significant correlation with TZ volume (P=0.08). Subtraction of BPSA from free PSA reduced its correlation with TZ volume to below that of PSA (from 0.64 to 0.48). Linear regression analyses showed that unlike PSA, both BPSA and free PSA display an age-independent relationship to TZ volume. ROC curve (for TZ>30cc) and binary logistic regression analyses showed that BPSA (AUC=0.844) outperforms both free PSA (AUC=0.799) and PSA (AUC=0.749) in its ability to predict clinically significant TZ enlargement.

Conclusion

In patients without prostate cancer, the serum concentration of BPSA displays an age-independent, log-linear relationship to TZ volume, and is a better predictor of prostate enlargement than PSA or free PSA. BPSA may also predict clinical parameters of BPH.

### EXAMPLE 8

### Comparison of the Percent of Different Molecular Forms of PSA for Prostate Cancer Detection in Men with Total Serum PSA Concentrations Between 4 and 10 ng/nl

Distinct molecular forms of free PSA (fPSA) have been identified. [−2]pPSA, a truncated form of the precursor of PSA that contains only 2 of the 7 leader amino acids, is produced primarily by prostate cancer tissue, while BPSA is found primarily in the transition zone of prostates exhibiting BPH. This study evaluates the performance of these two molecules for prostate cancer screening were evaluated.

Methods

Serum was prospectively collected from 217 consecutive patients who underwent ≧10 core transrectal, ultrasound-guided biopsy of the prostate and had a PSA between 4 and 10 ng/ml. 202 specimens were randomly selected from this pool for measurement of BPSA and 157 for [−2]pPSA. Hybritech Tandem-MP assays were used for measuring PSA and % fPSA. Serum [−2]pPSA and BPSA were measured with a research-only immunoassay developed at Beckman Coulter. The performance of the various markers was evaluated by ROC curve and binary logistic regression analyses using SPSS 10.0 (SPSS, Inc., Chicago, Ill.) statistical software.

Results

The median PSA, [−2]pPSA/fPSA, [−2]pPSA/BPSA, % BPSA, and % fPSA were 5.9 ng/ml, 0.5, 0.16, 3.89, and 13.6. BPSA and [−2]pPSA comprised, on average, 34.7 and 5.7% of free PSA respectively. ROC curve analysis demonstrated a non-statistically significant trend towards improved performance of both [−2]pPSA/fPSA (AUC=0.639 95% CI=0.554-0.719) and [−2]pPSA/BPSA (AUC=0.637 95% CI=0.551-0.717) as compared to either % fPSA (AUC=0.606 95% CI=0.520-0.688) or % BPSA (AUC=0.603 95% CI=0.516-0.685). Subtraction of BPSA from free PSA resulted in a statistically significant decrease in the performance of % fPSA (decrease in AUC from 0.606 to 0.534, P=0.049). Subtraction of [−2]pPSA from free PSA resulted in a statistically significant increase in the performance of % fPSA (increase in AUC from 0.606 to 0.616, P=0.025). Binary logistic regression analysis demonstrates that both % fPSA and % BPSA are independent predictors relative to [−2]pPSA/fPSA and [−2]pPSA/BPSA, and vise-versa, for the presence of cancer on biopsy.

Conclusions

The performance of % fPSA is driven by the BPSA component as evidenced by the decrease in performance of % fPSA when BPSA is subtracted from free PSA and the increase in performance when [−2]pPSA is subtracted. This agrees with published studies suggesting that the performance of % fPSA depends on its correlation with prostate volume. [−2]pPSA/fPSA may outperform % fPSA for prostate cancer screening in men with PSA concentrations of 4-10 ng/ml.

### EXAMPLE 9

### Comparison of the Percent of Different Molecular Forms of PSA for the Detection of Clinically Significant Prostate Cancer in Men with Total Serum PSA Concentrations Between 4 and 10 ng/nl

Distinct molecular forms of free PSA (fPSA) have been identified. [−2]pPSA, a truncated form of the precursor of PSA that contains only 2 of the 7 leader amino acids, is produced primarily by prostate cancer, while BPSA is found primarily in the transition zone of prostates exhibiting BPH. The performance of these two markers for the detection of clinically significant prostate cancer has been evaluated.

Methods

Serum was prospectively collected from 217 consecutive patients who underwent ≧10 core transrectal, ultrasound-guided biopsy of the prostate and had a PSA of 4-10 ng/ml. 202 specimens were randomly selected from this pool for measurement of BPSA and 157 for [−2]pPSA. Hybritech Tandem-MP assays were used for measuring PSA and % fPSA. Serum [−2]pPSA and BPSA were measured with an immunoassay developed at Beckman Coulter. The performance of the various markers was evaluated by ROC curve and binary logistic regression analyses using SPSS 10.0 (SPSS, Inc., Chicago, Ill.) statistical software.

Results

ROC curve analysis demonstrated a non-statistically significant trend towards improved performance of [−2]pPSA/fPSA (AUC=0.647 95% CI =0.560-0.727), [−2]pPSA/BPSA (AUC=0.668 95% CI=0.582-0.747), and % BPSA (AUC=0.646 95% CI=0.559-0.727) as compared to % fPSA (AUC=0.621 95% CI=0.448-0.622) for the detection of clinically significant prostate cancer (as defined by either more than one positive core or >2 mm of cancer or Gleason's grade>3). Subtraction of BPSA from free PSA resulted in a statistically significant decrease in the performance of % fPSA (decrease in AUC from 0.621 to 0.529, P=0.003). Subtraction of [−2]pPSA from free PSA resulted in a non-statistically significant increase in the performance of % fPSA (increase in AUC from 0.621 to 0.628, P=0.173). Binary logistic regression analysis demonstrates that the best predictors for the presence of clinically significant prostate cancer on biopsy, as opposed to either non-significant cancer or negative biopsy, are [−2]pPSA/fPSA and % BPSA.

Conclusions

The performance of % fPSA for the prediction of clinically significant prostate cancer is driven by the BPSA component as evidenced by the decrease in performance of % fPSA when BPSA is subtracted from free PSA and the increase in performance when [−2]pPSA is subtracted. [−2]pPSA/fPSA and % BPSA may outperform % fPSA for the detection of clinically significant prostate cancer in men with PSA between 4 and 10 ng/ml.

### EXAMPLE 10

### A Nomogram to Predict BPH-Related Surgery with or without Medical Therapy in Men with BPH

The purpose of this study was to develop a prediction model, or nomogram, that would predict the probability that a man with benign prostatic hyperplasia (BPH)-associated lower urinary tract symptoms (LUTS) would experience acute urinary retention (AUR) or require surgical intervention (SI) within 4 years, with or without medical therapy (finasteride and/or doxazosin).

Methods

Intent-to-treat, competing risks methodology was employed to model the 3,047 men with LUTS and BPH randomized to the MTOPS trial, a 5 year, randomized, placebo-controlled study evaluating the efficacy and safety of BPH medical therapy. These men were characterized at baseline by a number of parameters, including patient age and race, AUA symptom index (AUA-SI), BPH Impact Index (BII), total prostate and TZ volume measured by ultrasound, total prostate specific antigen (PSA) level, maximum flow rate (Qmax) and post-void residual (PVR) urine volume. Cox proportional hazards regression was used to relate these baseline variables to their future probability of AUR/SI within 4 years. The nomogram was internally validated with bootstrapping to assess its discrimination and calibration. Discrimination was quantified as the concordance index (CI).

Results

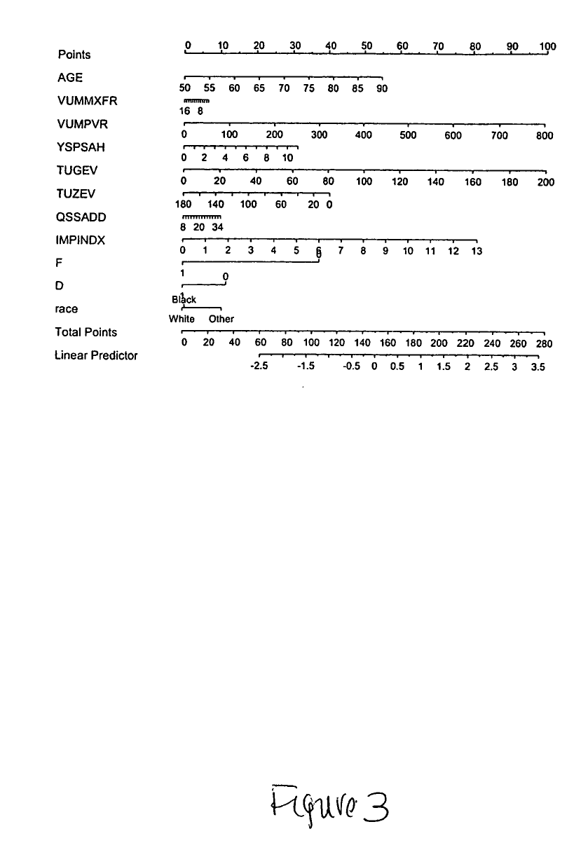

The nomogram (FIG. 3) appeared to be accurately calibrated and discriminating (CI=0.764, P<0.001). The table contains the p-values for the predictors in the multivariable model.

TABLE 11

| Variable | Description | Hazard Ratio | p-value |
|---|---|---|---|
| Age | Age | 1.0389649 | 0.005 |
| VUMMXFR | Qmax | 0.9847155 | 0.690 |
| VUMPVR | PVR | 1.0034764 | <0.001 |
| YSPSAH | total PSA | 1.0828093 | 0.086 |
| TUGEV | US total gland vol | 1.0140436 | 0.580 |
| TUZEV | US TZ volume | 0.9937542 | 0.600 |
| QSADD | AUA-SI | 1.0104136 | 0.580 |
| IMPINDX | BPH Impact Index | 1.1904294 | <0.001 |
| F | FIN randomization | 0.3505834 | <0.001 |
| D | DOX randomization | 0.7153833 | 0.086 |
| RACE = BLACK | | 1.0158497 | 0.960 |
| RACE = OTHER | | 1.3554489 | 0.340 |

Conclusion

A nomogram was constructed for predicting the probability that a man with a BPH-associated LUTS will experience AUR or require SI within 4 years, an outcome that appears highly predictable using readily available baseline clinical parameters. As expected finasteride, but not doxazosin, treated patients experienced a reduced risk of AUR and/or SI. Surprisingly, the BII at baseline, a relatively underutilized parameter compared to the AUA-SI, was a significant predictor of this endpoint, while AUA-SI was not. Furthermore, PVR but not Qmax also predicted AUR and/or SI. Finally, models that include US volume measurements appear to obviate the importance of total PSA as a predictor, presumably because of the high correlation between PSA and prostate volume parameters.

### EXAMPLE 11

### A Nomogram to Predict Symptom Progression with or without Medical Therapy in Men with BPH

The purpose of this study was to develop a prediction model, or nomogram, that would predict the probability that a man with benign prostatic hyperplasia (BPH)-associated lower urinary tract symptoms (LUTS) would experience symptom progression as defined in the MTOPS trial as a 4-point rise in AUA-SI from baseline, and later confirmed, within 4 years, with or without medical therapy (finasteride and/or doxazosin).

Methods

Intent-to-treat, competing risks methodology was used to model the 3,047 men with LUTS and BPH randomized to the MTOPS trial, a 5 year, randomized, placebo-controlled study evaluating the efficacy and safety of BPH medical therapy. These men were characterized at baseline by a number of parameters, including patient age and race, AUA symptom index (AUA-SI), BPH Impact Index (BII), total prostate and TZ volume measured by ultrasound, total prostate specific antigen (PSA) level, maximum flow rate (Qₘₐₓ), and post-void residual (PVR) urine volume. Cox proportional hazards regression was used to relate these baseline variables to their future probability of reaching a symptom progression endpoint within 4 years. The nomogram was internally validated with bootstrapping to assess its discrimination and calibration. Discrimination was quantified as the concordance index (CI).

Results

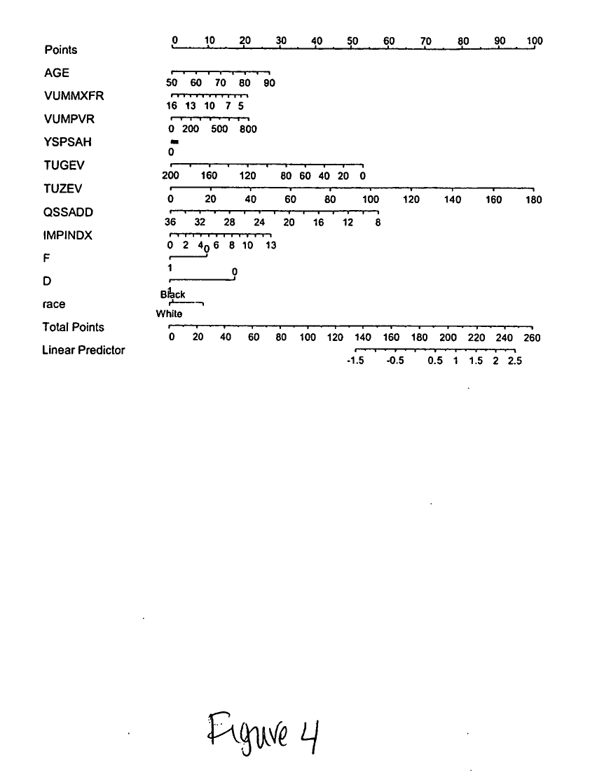

The nomogram (FIG. 4) appeared to be accurately calibrated and discriminating (CI=0.66, P<0.001). The table (Table 12) contains the p-values for the predictors in the multivariable model.

TABLE 12

| Description | Hazard Ratio | p-value |
|---|---|---|
| Age | 1.024335 | 0.008 |
| Qmax | 0.9395769 | 0.010 |
| PVR | 1.0009509 | 0.140 |
| total PSA | 1.0059789 | 0.860 |
| US total gland vol | 0.9905818 | 0.180 |
| US TZ volume | 1.0198269 | 0.029 |
| AUA-SI | 0.9296224 | <0.001 |
| BPH Impact Index | 1.0790012 | 0.009 |
| FIN randomization | 0.6960932 | 0.004 |
| DOX randomization | 0.5278221 | <0.001 |
| African American vs. Caucasian | 1.0317459 | 0.890 |
| Any other race vs. Caucasian | 1.9368852 | 0.100 |

Conclusion

A nomogram was constructed for predicting the probability that a man with a BPH-associated LUTS will experience symptom progression within 4 years. This outcome appears less predictable than a retention-surgery endpoint (CI 0.764 vs. 0.66) using readily available baseline clinical parameters. As expected, both finasteride and doxazosin-treated patients experienced a significantly reduced risk of symptom progression. Also as noted previously, lower baseline AUA-SI was associated with a higher risk of symptom progression, likely due to the “regression to the mean” phenomenon. Finally, baseline PSA level provides little if any independent value for predicting symptom progression in these patients.

### EXAMPLE 12

### Inclusion of BPSA in Risks Model for AUR or SI in BPH Patients

Table 13 provides P values for a series of predictors, including BPSA levels, in a multivariate competing risks model for AUR and/or SI. BPSA is a novel serum marker for BPH, and independently predicts the risk of AUR and/or surgery in men with BPH. Thus, nomogram tools that incorporate BPSA levels may improve the ability to manage patients with BPH.

TABLE 13

| Predictor | Chi-Square | d.f. | P value |
|---|---|---|---|
| Age | 5.30 | 1 | 0.0213 |
| Qmax | 0.32 | 1 | 0.5694 |
| PVR | 32.95 | 2 | <.0001 |
| Nonlinear | 7.14 | 1 | 0.0075 |
| US Total Pros Vol | 1.39 | 1 | 0.2378 |
| US TX Pros Vol | 0.25 | 1 | 0.6160 |
| AUA-SI | 0.73 | 1 | 0.3919 |
| BPH Impact Index | 20.02 | 1 | <.0001 |
| Total PSA | 3.23 | 2 | 0.199 |
| Nonlinear | 3.06 | 1 | 0.0801 |
| Free PSA | 2.51 | 1 | 0.1134 |
| ProPSA | 0.01 | 1 | 0.915 |
| BPSA | 4.54 | 1 | 0.0331 |
| Finasteride | 16.68 | 1 | <.0001 |
| Doxazosin | 4.09 | 1 | 0.0431 |

### REFERENCES

Arrighi et al., Urology, 381:4 (1991).
Berry et al., J. Urol., 132:474 (1984).
Concato et al., Journal of Clinical Epidemiology, 48:1495-501 (1995).
Cox, J. Royal Statistical Society, B34, 187-220 (1972).
Danielpour et al., J. Cell. Phy., 138:79 (1989).
Danielpour et al., J. Immunol. Meth., 148:17 (1993).
Dasch et al., Annals New York Academy of Sciences, pp. 303-305 (1990).
Eastham et al., Sem. Urol. Oncol., 20:108 (2002).
Efron et al., An Introduction to the Bootstrap, Chapman & Hall, New York, N.Y. (1993).
Emberton et al., Urology, 61:267 (2002).
Flanders, J. Cell Biol., 108:653 (1989).
Haese et al., J. Urol., 158:2188 (1997).
Jacobsen et al., J. Urol., 155:595 (1999).
Jacobsen et al., J. Urol., 158:481 (1997).
Jacobsen et al., J. Urol., 162:1301 (1999).
Kattan et al., J. Urol., 170:56 (2003).
Kattan et al., Journal of Urology, 169:475 (2003).
Lilja et al., Clin. Chem., 37:1618 (1991).
Linton et al., Clinical Chemistry, 49:253 (2003).
Lopez-Corona et al., J. of Urol., 170:1184 (2003).
Lucas et al., J. Immunol., 145:1415 (1990).
Maru et al., Urology submitted (2003).
McConnell et al., N. Engl. J. Med., 349:2387 (2003).
McConnell et al., N. Engl. J. Med., 338:557 (1998).
Mettlin et al., Prostate, 39:153 (1999).
Mikolajczyk et al., Cancer Res., 61:6958 (2001).
Mikolajczyk et al., Cancer Research, 60:756 (2000).
Mikolajczyk et al., Prostate, 45:271 (2000).
Mikolajczyk et al., Urology, 50:710 (1997).
Mikolajczyk et al., Urology, 55:41 (2000).
Morote et al., Eur. Urol., 38:91 (2000).
Prakash et al., PNAS, 99:7598 (2002).
   Prevention and Cure of Prostate Cancer (1998).
Roehrborn et al., Eur. J. Urol., 42:1 (2002).
Roehrborn et al., Urology, 54:662 (1999).
Roehrborn et al., J. Urol., 163:13 (2000).
Roehrborn et al., Urology, 53:473 (1999).
Roehrborn et al., Urology, 53:581 (1999).
Roehrborn et al., Urology, 60:434 (2002).
Slawin et al., AACR Special Conference, New Research Approaches in the
Sokoll et al., Urol., 61:274 (2003).
Song et al., J. Urol., Abstract (1997).
Stamey et al., Clin. Chem., 47:631 (2001).
Stenman et al., Cancer Res., 51:222 (1991).
Stephan et al., Cancer, 79:104 (1997).
Thompson et al., J. Cell Biol., 108:661 (1989).
Wang et al., Eur. J. Biochem., 267:4040 (2000).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to predict the probability of acute urinary retention (AUR), surgical intervention (SI), or symptom progression in a patient with benign prostatic hyperplasia (BPH), comprising:
a) providing a value for a plurality of patient factors including age, prostate specific antigen (PSA) level, post-void residual urine volume (PVR), maximal flow rate of urine (Qmax), prostate volume (PV), American Urological Association symptom index (AUA-SI) score, BPH impact index (BII) score, benign PSA (BPSA) level, non alpha blocker BPH drug therapy, and/or prior use of an alpha blocker; and
b) correlating the values for the plurality of factors with the probability of AUR, SI, or symptom progression in the patient.

2. The method of claim 1 wherein the probability of AUR or SI is within 2 years.

3. The method of claim 1 wherein the probability of AUR or SI is within 4 years.

4. The method of claim 1 wherein the plurality of factors includes age, PVR and BII score, and optionally non alpha blocker BPH drug therapy, PSA level, and/or PV.

5. The method of claim 1 wherein the plurality of factors includes age, AUA-SI score, BII score, Qmax, and non alpha blocker BPH drug therapy, and optionally PVR and prostate transition zone volume.

6. The method of claim 1 wherein the plurality of factors includes BII score, PV, PSA level, Qmax, non alpha blocker BPH drug therapy, and prior alpha blocker use, and optionally AUA-SI score.

7. The method of claim 1 wherein the plurality of factors includes PVR, BII score, and non alpha blocker BPH drug therapy, and optionally BPSA level.

8. The method of claim 1 wherein prior use of an alpha blocker is not a factor.

9. The method of claim 4 or 6 wherein the values for the plurality of factors are correlated to the probability of AUR and/or SI.

10. The method of claim 5 wherein the values for the plurality of factors are correlated to the probability of symptom progression.

11. The method of any one of claims 1 to 10 wherein the correlating is conducted by a computer.

12. The method of claim 1 wherein the non alpha blocker BPH drug is a 5 alpha reductase inhibitor.

13. An apparatus, comprising:
a data input means, for input of information for a plurality of factors in a mammal having BPH, factors including age, PSA level, PVR, PV, Qmax, AUA-SI score, BII score, BPSA level, non alpha blocker BPH drug therapy, and/or prior use of an alpha blocker;
a processor, executing a software for analysis of the information;
wherein the software analyzes the information and provides the probability of AUR, SI or symptom progression in the mammal.

14. The apparatus of claim 13 wherein the plurality of factors includes age, PVR, and BII score, and optionally PSA level, PV, and/or non alpha blocker BPH drug therapy.

15. The apparatus of claim 13 wherein the plurality of factors includes age, AUA-SI score, BII score, Qmax, and non alpha blocker BPH drug therapy, and optionally PVR and/or prostate transition zone volume.

16. The apparatus of claim 13 wherein the plurality of factors includes BII score, PV, PSA level, Qmax, non alpha blocker BPH drug therapy, and prior alpha blocker use, and optionally AUA-SI score.

17. The apparatus of claim 13 wherein the plurality of factors includes PVR, BII score, and non alpha blocker BPH drug therapy, and optionally BPSA level.

18. The apparatus of claim 13 wherein the factors are input manually using the data input means.

19. The apparatus of claim 13 wherein the software constructs a database of the information.

20. The apparatus of claim 13 wherein the non alpha blocker BPH drug is a 5 alpha reductase inhibitor.

21. The apparatus of claim 13 wherein prior use of an alpha blocker is not a factor.

22. A method to predict the probability of AUR, SI or symptom progression in a patient with BPH, comprising:
a) inputting information to a data input means, wherein the information comprises a plurality of factors including age, PSA level, PVR, PV, Qmax, AUA-SI score, BII score, BPSA level, non alpha blocker BPH drug therapy, and/or prior use of an alpha blocker;
b) executing a software for analysis of the information; and
c) analyzing the information so as to provide the probability of AUR, SI or symptom progression in the patient.

23. The method of claim 22 wherein the information comprises the following factors: age, PVR, and BII score, and optionally PSA level, PV, and non alpha blocker BPH drug therapy.

24. The method of claim 22 wherein the information comprises the following factors: age, AUA-SI score, BII score, Qmax, and non alpha blocker BPH drug therapy, and optionally PVR and/or prostate transition zone volume.

25. The method of claim 22 wherein the information comprises the following factors BII score, PV, PSA level, Qmax, non alpha blocker BPH drug therapy, and prior alpha blocker use, and optionally AUA-SI score.

26. The method of claim 22 wherein the information comprises the following factors PVR, BII score, and non alpha blocker BPH drug therapy, and optionally BPSA level.

27. The method of claim 22 wherein the non alpha blocker BPH drug is a 5 alpha reductase inhibitor.

28. The method of claim 22 wherein prior use of an alpha blocker is not a factor.

29. A nomogram for the graphic representation of a quantitative probability of AUR, SI, or symptom progression in a BPH patient, comprising: a plurality of scales and a solid support, the plurality of scales being disposed on the support and comprising a scale for a plurality of factors including age, PSA level, PVR, PV, Qmax, AUA-SI score, BII score, BPSA level, non alpha blocker BPH drug therapy, and/or prior use of an alpha blocker, a points scale, a total points scale and a predictor scale, wherein the scales for age, PSA level, PVR, Qmax, PV, AUA-SI score, BII score, BPSA level, non alpha blocker BPH drug therapy, and/or prior use of an alpha blocker, each has values on the scales, and wherein the scales for age, PSA level, PVR, Qmax, PV, AUA-SI score, BII score, BPSA level, non alpha blocker BPH drug therapy, and/or prior use of an alpha blocker are disposed on the solid support with respect to the points scale so that each of the values for age, PSA level, PVR, PV, Qmax, AUA-SI score, BII score, BPSA level, non alpha blocker BPH drug therapy, and/or prior use of an alpha blocker can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the patient's age, PSA level, PVR, Qmax, PV, AUA-SI score, BII score, BPSA level, non alpha blocker BPH drug therapy, and/or prior use of an alpha blocker can be added together to yield a total points value, and the total points value can be correlated with the predictor scale to predict the quantitative probability of AUR, SI, or symptom progression.

30. The nomogram of claim 29 which is a graphic representation of a quantitative probability of AUR or SI within 2 years.

31. The nomogram of claim 29 which is a graphic representation of a quantitative probability of AUR or SI within 4 years.

32. The nomogram of claim 29 wherein the scales are for age, PVR and BII score, and optionally non alpha blocker BPH drug therapy, PSA level, and/or PV.

33. The nomogram of claim 29 wherein the scales are for age, AUA-SI score, BII score, Qmax, and non alpha blocker BPH drug therapy, and optionally PVR and volume of the prostate transition zone.

34. The nomogram of claim 29 wherein the scales are for BII score, PV, PSA level, Qmax, non alpha blocker BPH drug therapy, and prior alpha blocker use, and optionally AUA-SI score.

35. The nomogram of claim 29 wherein the scales are for PVR, BII score, and non alpha blocker BPH drug therapy, and optionally BPSA level.

36. The nomogram of claim 29 wherein the non alpha blocker BPH drug is a 5 alpha reductase inhibitor.

37. The nomogram of claim 29 wherein prior use of an alpha blocker is not a factor.

38. The nomogram of claim 29 wherein the solid support is a laminated card.

39. A method to predict the probability of AUR and/or SI in a BPH patient comprising: providing a factor value for each of a set of factors for a patient, which factors include age, PSA level, BPSA level, PV, PVR, Qmax, AUA-SI score, BII score, non alpha blocker BPH drug therapy, and/or prior use of an alpha blocker; determining a separate point value for each of the factor values using the nomogram of claim 29; adding the separate point values together to yield a total points value; and correlating the total points value with a value on the predictor scale of the nomogram to determine the probability of AUR and/or SI for the patient.

40. A method to predict BPH symptom progression in a BPH patient comprising: providing a factor value for each of a set of factors for a patient, which factors include age, AUA-SI score, BII score, Qmax, PVR, volume of the prostate transition zone, and/or non alpha blocker BPH drug therapy; determining a separate point value for each of the factor values using the nomogram of claim 29; adding the separate point values together to yield a total points value; and correlating the total points value with a value on the predictor scale of the nomogram to determine the probability of symptom progression in the patient.

41. The method of claim 39 or 40 wherein the correlating is conducted by a computer.

42. The method of claim 39 or 40 wherein the non alpha blocker BPH drug is a 5 alpha reductase inhibitor.

43. The method of claim 39 or 40 wherein prior use of an alpha blocker is not a factor.

44. A method to predict prostate enlargement in a male without prostate cancer, comprising:
a) detecting or determining BPSA levels in a physiological fluid sample from a male without prostate cancer; and
b) correlating BPSA levels with the probability of an enlarged prostate.

45. The method of claim 40 wherein the BPSA levels are correlated with the probability of an enlarged transition zone.

46. A system comprising:
a nomogram database including data representative of nomograms useful to predict ailment progression;
software operable on the system to:
receive data representative of a plurality of patient factors including a diagnosed patient ailment from a client;
retrieve nomogram data values from the nomogram database based on the plurality of patient factors; and
correlate the nomogram data values for the plurality of patient factors with a probability of ailment progression.

47. The system of claim 46, wherein the ailment is benign prostatic hyperplasia (BPH).

48. The system of claim 46, further comprising:
a network connection device; and
wherein the data representative of a plurality of patient factors is received from the client over the network connection device.

49. The system of claim 48, wherein the software is further operable on the system to:
communicate the probability of ailment procession to the client over the network connection device.

50. The system of claim 48, wherein the network connection device can be operatively coupled to the Internet.
